SEQUENCE LISTING (3 sequences)

SEQ ID NO: 1 - DNA, 1128 bp, HSV1
atggcttcgtacccctgccatcaacacgcgtctgcgttcgaccaggctgcgcgttctcgcggccataacaaccgacgtacggcgttgcgccctcgccggcagcaaaaagccacggaagtccgcctggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgttacccggccgcgcgataccttatgggcagcatgaccccccaggccgtgctggcgttcgtggccctcccgccgaccttgcccggcacaaacatcgtgttgggggcccttccggaggacagacacatcgaccgcctggccaaacgccagcgccccggcgagcggcttgacctggctatgctggccgcgattcgccgcgtttacgagctgcttgccaatacggtgcggtatctgcagggcggcgggtcgtggcgggaggattggggacagctttcggggacggccgtgccgccccagggtgccgagccccagagcaacgcgggcccacaaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactaa

SEQ ID NO: 2 - protein, 375 aa, HSV1
MASYPCHQHASAFDQAARSRGHNNRRTALRPRRQQKATEVRLEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALPPTLPGTNIVLGALPEDRHIDRLAKRQRPGERLDLAMLAAIRRVYELLANTVRYLQGGGSWREDWGQLSGTAVPPQGAEPQSNAGPQPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 3 - DNA, 1145 bp, HSV1
atggcttcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccatcttcctcgaccgccatcccatcgccttcatgctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgacccggggaattccg

(19) United States
(12) Patent Application Publication
(10) Pub. No.: US 2010/0322861 A1
(43) Pub. Date: Dec. 23, 2010

(54) ENGINEERED CELLS, IMAGING REPORT GENE/PROBE SYSTEMS, AND METHODS OF IMAGING

(76) Inventors: Sanjiv S. Gambhir, Portola Valley, CA (US); Shahriar Yaghoubi, Beverly Hills, CA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/744,386

(22) PCT Filed: Nov. 26, 2008
(86) PCT No.: PCT/US08/84785

§ 371 Date: May 24, 2010

Related U.S. Application Data
(60) Provisional application No. 60/990,329, filed on Nov. 27, 2007.

(51) Int. Cl. A61K 49/00; C12N 5/10

(52) U.S. Cl. 424/9.1; 435/325

(57) ABSTRACT

Embodiments of the present disclosure provide: methods of imaging the location and survival of an engineered cell in a host (e.g., human) with an imaging reporter probe, methods of imaging the location and survival of an engineered cell in a host, and, kits, engineered cells, and methods of making the engineered cells, and the like.

### CROSS-REFERENCE TO RELATED APPLICATION

This application claims priority to U.S. provisional application entitled, “IMAGING REPORTER GENE/PROBE SYSTEMS AND METHODS OF IMAGING,” having Ser. No. 60/990,329, filed on Nov. 27, 2007, which is entirely incorporated herein by reference.

### STATEMENT REGARDING FEDERALLY SPONSORED RESEARCH OR DEVELOPMENT

This invention was made with government support under Grants: R01 CA135486 and National Institutes of Health (NIH) NCI ICMIC P50. The government has certain rights in the invention.

### BACKGROUND

In general, non-invasive imaging of administered cells to a human has involved inserting an imaging probe into cells ex vivo prior to their administration. In some instances, the probes are then imaged using a nuclear imaging technique (e.g., SPECT). One disadvantage of this approach is that cell division dilutes the amount of probes in each cell. Another disadvantage is that when the cells die, the probes are released and would emit a signal providing a misleading signal. Furthermore, radionuclides have a certain half life, which does not allow long term imaging of cells. Therefore, other alternative non-invasive imaging techniques are needed.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A is a diagram of an embodiment of an engineered cell and the cycle of the imaging reporter probe.

FIG. 1B is a structure of an embodiment of an imaging reporter probe.

FIG. 2 illustrates digital MRI images and PET over MRI superimposed brain images of a patient who had been infused with autologous cytolytic T cells expressing IL13 zetakine and HSV1-tk genes. Images were acquired approximately two hours after [¹⁸F]FHBG injection. The patient had a surgically ressected tumor (1) in the left corner and a new non-ressected tumor in the center (2), near the corpus callosum of his brain. The infused cells had localized at the site of tumor 1 and also trafficked to tumor 2. [¹⁸F]FHBG activity is higher than the brain background at both sites. Background [¹⁸F]FHBG activity is low within the central nervous system due to its inability to cross the blood brain barrier. Background activity is relatively higher in all other tissues. Activity can also be observed in the meninges. The tumor 1/meninges and tumor 2/meninges [¹⁸F]FHBG activity ratios in this patient were 1.75 and 1.57, respectively. The average resected tumor site/meninges and intact tumor site to meninges [¹⁸F]FHBG activity ratio in control patients was 0.86 and 0.44, respectively.

FIGS. 3 and 4 illustrate whole-body digital PET images and PET/CT images of [¹⁸F]FHBG biodistribution in a human, two hours after intravenous injection. Two coronal slices are shown to illustrate activity within the liver, gall-bladder, intestines, kidneys and bladder, which are organs involved with [¹⁸F]FHBG's clearance from the body. Background activity in all other tissues is relatively low, due to the absence of HSV1-tk or HSV1-sr39tk expressing cells within the body of this human volunteer.

FIG. 5 is a bar graph that illustrates the uptake of [³H]Penciclovir into the genetically engineered CTL clone that was infused into the patient and control Jurkat cells. The autologous patient CTLs were genetically engineered to constitutively express HSV1-tk. The control cells expressed Firefly luciferase instead of HSV1-tk. Whereas control cells had a fixed uptake at both 1 h and 4 h, the CTL clone had a 2.7× higher uptake at 1 h and 9.3× higher uptake at 4 h. The symbol “*” indicates significant difference between uptake of [³H]PCV into patient CTLs vs control cells at 4 hours (P<0.001).

FIG. 6 illustrates Table 1.

FIGS. 7A-7D illustrate tables including vital signs information about the patient.

FIG. 8 illustrates a table describing quality assurance criteria of infused CTLs.

FIG. 9 illustrates a graph of the percent injected dose per gram of plasma or blood cells versus minutes after probe injection for blood cells and plasma.

FIG. 10 illustrates a Time-Line of Events of the study described in the Example below.

### SUMMARY

Embodiments of the present disclosure provide: methods of imaging the location and survival of an imaging reporter gene expressing cell in a host (e.g., human) with an imaging reporter probe, methods of imaging the location and survival of an imaging reporter gene expressing cell in a human, and kits, engineered cells, and methods of making engineered cells of the present disclosure, and the like.

Embodiments of the method of imaging the location and survival of an engineered cell in a human with an imaging reporter probe, among others, include: delivering to the human subject an engineered cell, wherein the engineered cell includes an imaging reporter gene, and wherein the imaging reporter gene is expressed in the cell, thereby generating an imaging reporter gene product; administering to the human subject an imaging reporter probe, wherein the imaging reporter probe freely enters and exits the cell, wherein the imaging reporter gene product interacts with the imaging reporter probe to form a modified imaging reporter probe, wherein the modified imaging reporter probe accumulates either within the engineered cell or on the surface of the engineered cell that expresses the imaging reporter gene, wherein the imaging reporter probe has a characteristic that it is capable of being imaged non-invasively using a nuclear imaging system, magnetic resonance imaging system, or an optical imaging system in the human; and non-invasively imaging the human subject, wherein detecting the presence of the modified imaging reporter probe corresponds to the presence of the engineered cell.

Embodiments of the method of imaging the location and survival of an engineered cell in a human, among others, include: delivering to the human subject an engineered cell, wherein the engineered cell includes an imaging reporter gene, and wherein the imaging reporter gene is expressed in the engineered cell, thereby generating an imaging reporter gene product, wherein the imaging reporter gene product accumulates either within the engineered cell or on the surface of the engineered cell that expresses the imaging reporter gene, wherein the imaging reporter gene product has a characteristic that it is capable of being imaged non-invasively using a nuclear imaging system, magnetic resonance imaging system, or an optical imaging system in the human; and non-invasively imaging the human subject, wherein detecting the presence of the imaging reporter gene product corresponds to the presence of the engineered cell.

Embodiments of the method of imaging the location and survival of an engineered cell in a human with an imaging reporter probe, among others, include: delivering to the human subject an engineered cell, wherein the engineered cell includes an imaging reporter gene; administering to the human subject an imaging reporter probe, wherein the imaging reporter probe freely enters and exits the cell, wherein the imaging reporter gene interacts with the imaging reporter probe so that the imaging reporter probe accumulates either within the engineered cell or on the surface of the engineered cell, wherein the imaging reporter probe has a characteristic that it is capable of being imaged non-invasively using a nuclear imaging system, magnetic resonance imaging system, or an optical imaging system in the human; and non-invasively imaging the human subject, wherein detecting the presence of the imaging reporter probe corresponds to the presence of engineered cell.

Embodiments of the kit, among others, include: an engineered cell, wherein the engineered cell includes an imaging reporter gene, and wherein the imaging reporter gene is expressed in the cells, thereby generating an imaging reporter gene product; an imaging reporter probe; and directions for use.

Embodiments of the kit, among others, include: an engineered cell, wherein the engineered cell includes an imaging reporter gene, and wherein the imaging reporter gene is expressed in the cells, thereby generating an imaging reporter gene product, wherein the imaging reporter gene product accumulates either within the cells or on the surface of the cells that express the imaging reporter gene, wherein the imaging reporter probe has a characteristic that it is capable of being imaged non-invasively using a nuclear imaging system, magnetic resonance imaging system, or an optical imaging system in the human; and directions for use.

Embodiments of the engineered cell, among others, include: an imaging reporter gene, wherein the imaging reporter gene is expressed in the cells, thereby generating an imaging reporter gene product.

Embodiments of the engineered cell, among others, include: an imaging reporter gene, wherein the imaging reporter gene is expressed in the cells, thereby generating an imaging reporter gene product, wherein the imaging reporter gene product accumulates either within the cells or on the surface of the cells that express the imaging reporter gene, wherein the imaging reporter gene product has a characteristic that it is capable of being imaged non-invasively using a nuclear imaging system, magnetic resonance imaging system, or an optical imaging system in the human.

Embodiments of the method of imaging the location and survival of an engineered cell in a host with an imaging reporter probe, among others, include: delivering to the host an engineered cell, wherein the engineered cell includes an imaging reporter gene, and wherein the imaging reporter gene is expressed in the cell, thereby generating an imaging reporter gene product; administering to the host subject an imaging reporter probe, wherein the imaging reporter probe freely enters and exits the cell, wherein the imaging reporter gene product interacts with the imaging reporter probe to form a modified imaging reporter probe, wherein the modified imaging reporter probe accumulates either within the engineered cell or on the surface of the engineered cell that expresses the imaging reporter gene, wherein the imaging reporter probe has a characteristic that it is capable of being imaged non-invasively using a nuclear imaging system, magnetic resonance imaging system, or an optical imaging system in the host; and non-invasively imaging the host, wherein detecting the presence of the modified imaging reporter probe corresponds to the presence of the engineered cell.

Embodiments of the method of imaging the location and survival of an engineered cell in a host with an imaging reporter probe, among others, include: delivering to the host an engineered cell, wherein the engineered cell includes an imaging reporter gene, and wherein the imaging reporter gene is expressed in the engineered cell, thereby generating an imaging reporter gene product, wherein the imaging reporter gene product accumulates either within the engineered cell or on the surface of the engineered cell that expresses the imaging reporter gene, wherein the imaging reporter gene product has a characteristic that it is capable of being imaged non-invasively using a nuclear imaging system, magnetic resonance imaging system, or an optical imaging system in the host; and non-invasively imaging the host, wherein detecting the presence of the imaging reporter gene product corresponds to the presence of the engineered cell.

Embodiments of the method of imaging the location and survival of an engineered cell in a host with an imaging reporter probe, among others, include: delivering to the host an engineered cell, wherein the engineered cell includes an imaging reporter gene; administering to the host subject an imaging reporter probe, wherein the imaging reporter probe freely enters and exits the cell, wherein the imaging reporter gene interacts with the imaging reporter probe so that the imaging reporter probe accumulates either within the engineered cell or on the surface of the engineered cell, wherein the imaging reporter probe has a characteristic that it is capable of being imaged non-invasively using a nuclear imaging system, magnetic resonance imaging system, or an optical imaging system in the host; and non-invasively imaging the host, wherein detecting the presence of the imaging reporter probe corresponds to the presence of engineered cell.

These embodiments, uses of these embodiments, and other uses, features and advantages of the present disclosure, will become more apparent to those of ordinary skill in the relevant art when the following detailed description of the preferred embodiments is read in conjunction with the appended figures.

### DETAILED DESCRIPTION

Before the present disclosure is described in greater detail, it is to be understood that this disclosure is not limited to particular embodiments described, and that the various embodiments of the invention may, of course, vary. It is also to be understood that the terminology used herein is for the purpose of describing particular embodiments only, and is not intended to be limiting, because the scope of the present disclosure is to be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of biology, molecular biology, synthetic organic chemistry, biochemistry, and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### DEFINITIONS

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y., Current Protocols in Molecular Biology (Ausbel et al., eds., John Wiley & Sons, Inc. 2001), as well as other well known books and publications. Manufacturer defined protocols and/or parameters can be used as they relate to the use of commercially available kits and reagents.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

“Peptide” refers to a polymer in which the monomers are amino acid residues, which are joined together through amide bonds, alternatively referred to as a polypeptide. A “single polypeptide” is a continuous peptide that constitutes the protein. When the amino acids are alpha-amino acids, either the L-optical isomer or the D-optical isomer can be used, the L-isomers being preferred. Additionally, unnatural amino acids such as beta-alanine, phenylglycine, and homo-arginine are meant to be included. Commonly encountered amino acids, which are not gene-encoded can also be used herein, although preferred amino acids are those that are encodable.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified (e.g., hydroxyproline, gamma-carboxyglutamate, and O-phosphoserine). Amino acid analogs refers to compounds that have the same basic chemical structure as a naturally occurring amino acid (e.g., an a carbon that is bound to a hydrogen, a carboxyl group, an amino group, and an R group, e.g., homoserine, norleucine, methionine sulfoxide, methionine methyl sulfonium). Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but function in a manner similar to a naturally occurring amino acid.

“Amino acids” may be referred to herein by either their commonly known three letter symbols or by the one-letter symbols recommended by the IUPAC-IUB Biochemical Nomenclature Commission as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). Nucleotides, likewise, may be referred to by their commonly accepted single-letter codes as indicated as follows: Adenine (A), Guanine (G), Cytosine (C), Thymidine (T), and Uracil (U), which are discussed in more detail below.

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall (homologous) and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of this disclosure and still result in a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biologically functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala: Gly, Ser), (Arg: Lys), (Asn: Gin, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also refers to the degree of sequence relatedness between polypeptide as determined by the match between strings of such sequences. “Identity” and “similarity” can be readily calculated by known methods, including, but not limited to, those described in books and papers such as: Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073, (1988).

Preferred methods to determine identity are designed to give the largest match between the sequences tested. Methods to determine identity and similarity are codified in publicly available computer programs. The percent identity between two sequences can be determined by using analysis software that are known in the art (e.g., NBLAST, and XBLAST). The default parameters are used to determine the identity for the polypeptides used herein.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution (including conservative and non-conservative substitution), or insertion, and wherein said alterations may occur at the amino- or carboxy-terminus positions of the reference polypeptide sequence or anywhere between those terminal positions, interspersed either individually among the amino acids in the reference sequence, or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Conservative amino acid variants can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-naturally occurring amino acid residues into proteins, and a few are described below.

For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography.

In a second method, translation is carried out in Xenopus oocytes by microinjection of mutated mRNA and chemically aminoacylated suppressor tRNAs (Turcatti, et al., J. Biol. Chem., 271: 19991-8, 1996).

In a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. (Koide, et al, Biochem., 33: 7470-6, 1994). Naturally occurring amino acid residues can be converted to non-naturally occurring species by in vitro chemical modification. Chemical modification can be combined with site-directed mutagenesis to further expand the range of substitutions (Wynn, et al., Protein Sci., 2: 395-403, 1993).

The term “heterologous” when used with reference to portions of a nucleic acid indicates that the nucleic acid comprises two or more subsequences that are not found in the same relationship to each other in nature. For instance, a heterologous nucleic acid is typically recombinantly produced, having two or more sequences from unrelated genes arranged to make a new functional nucleic acid (e.g., a nucleic acid encoding a fluorescent protein from one source and a nucleic acid encoding a peptide sequence from another source). Similarly, a heterologous protein indicates that the protein comprises two or more subsequences that are not found in the same relationship to each other in nature (e.g., a fusion protein).

As used herein, the term “polynucleotide” generally refers to any polyribonucleotide or polydeoxyribonucleotide, which may be unmodified RNA or DNA or modified RNA or DNA. Polynucleotides can refer to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. It should also be noted that the terms “nucleic acid,” “nucleic acid sequence,” or “oligonucleotide” also encompass a polynucleotide as defined above.

In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

As used herein, DNA may be obtained by any method. For example, the DNA includes complementary DNA (cDNA) prepared from mRNA, DNA prepared from genomic DNA, DNA prepared by chemical synthesis, DNA obtained by PCR amplification with RNA or DNA as a template, and DNA constructed by appropriately combining these methods.

As used herein, an “isolated nucleic acid” is a nucleic acid, the structure of which is not identical to that of any naturally occurring nucleic acid or to that of any fragment of a naturally occurring genomic nucleic acid spanning more than three genes. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, e.g., a gene encoding a fusion protein. Specifically excluded from this definition are nucleic acids present in random, uncharacterized mixtures of different DNA molecules, transfected cells, or cell clones, e.g., as these occur in a DNA library such as a cDNA or genomic DNA library.

The term “substantially pure” as used herein in reference to a given polypeptide means that the polypeptide is substantially free from other biological macromolecules. For example, the substantially pure polypeptide is at least 75, 80, 85, 95, or 99% pure by dry weight. Purity can be measured by any appropriate standard method known in the art, for example, by column chromatography, polyacrylamide gel electrophoresis, or HPLC analysis.

The DNA encoding the proteins disclosed herein can be prepared by the usual methods known to those of skill in the art: cloning cDNA from mRNA encoding the protein, isolating genomic DNA and splicing it, chemical synthesis, and so on.

cDNA can be cloned from mRNA encoding the protein by, for example, the method described below:

First, the mRNA encoding the protein is prepared from tissues or cells expressing and producing the protein. mRNA can be prepared by isolating total RNA by a known method such as guanidine-thiocyanate method (Chirgwin et al., Biochemistry, 18:5294, 1979), hot phenol method, or AGPC method, and subjecting it to affinity chromatography using oligo-dT cellulose or poly-U Sepharose.

Then, with the mRNA obtained as a template, cDNA is synthesized, for example, by a well-known method using reverse transcriptase, such as the method of Okayama et al (Mol. Cell. Biol. 2:161 (1982); Mol. Cell. Biol. 3:280 (1983)) or the method of Hoffman et al. (Gene 25:263 (1983)), and converted into double-stranded cDNA. A cDNA library is prepared by transforming E. coli with plasmid vectors, phage vectors, or cosmid vectors having this cDNA or by transfecting E. coli after in vitro packaging.

The plasmid vectors used herein are not limited as long as they are replicated and maintained in hosts. Any phage vector that can be replicated in hosts can also be used. Examples of usually used cloning vectors are pUC19, gt10, gt11, and so on. When the vector is applied to immunological screening, as mentioned below, a vector having a promoter that can express a gene encoding the desired protein in a host is preferably used.

cDNA can be inserted into a plasmid by, for example, the method of Maniatis et al. (Molecular Cloning, A Laboratory Manual, second edition, Cold Spring Harbor Laboratory, p. 1.53, 1989). cDNA can be inserted into a phage vector by, for example, the method of Hyunh et al. (DNA cloning, a practical approach, 1, p. 49 (1985)). These methods can be simply performed by using a commercially available cloning kit (e.g., a product from Takara Shuzo). The recombinant plasmid or phage vector thus obtained is introduced into an appropriate host cell such as a prokaryote (e.g., E. coli: HB101, DH5a, MC1061/P3, etc).

Examples of a method for introducing a plasmid into a host (e.g., cell) include, calcium chloride method, calcium chloride/rubidium chloride method and electroporation method, described in Molecular Cloning, A Laboratory Manual (second edition, Cold Spring Harbor Laboratory, p. 1.74 (1989)). Phage vectors can be introduced into host cells by, for example, a method in which the phage DNAs are introduced into grown hosts after in vitro packaging. In vitro packaging can be easily performed with a commercially available in vitro packaging kit (for example, a product from Stratagene or Amersham). Genes can also be introduced into a host using viral and non-viral vectors.

The identification of cDNA encoding protein, its expression being augmented depending on the stimulation of cytokines like AID protein disclosed herein, can be carried out by, for example, suppression subtract hybridization (SSH)(Proc. Natl. Acad. Sci. USA, 93:6025-6030, 1996; Anal. Biochem., 240:90-97, 1996) taking advantage of suppressive PCR effect (Nucleic Acids Res., 23:1087-1088, 1995), using two cDNA libraries, namely, cDNA library constructed from mRNA derived from stimulated cells (tester cDNA library) and that constructed from mRNA derived from unstimulated cells (driver cDNA library).

Specific examples of the vectors for recombination used are E. coli-derived plasmids such as pBR322, pBR325, pUC12, pUC13, and pUC19, yeast-derived plasmids such as pSH19 and pSH15, and Bacillus subtilis-derived plasmids such as pUB110, pTP5, and pC194. Examples of phages are a bacteriophage such as phage (e.g., gamma), and an animal or insect virus (pVL1393, Invitrogen) such as a retrovirus, a vaccinia virus, and a nuclear polyhedrosis virus.

An “expression vector” is useful for expressing the DNA encoding the protein used herein and for producing the protein. The expression vector is not limited as long as it expresses the gene encoding the protein in various prokaryotic and/or eukaryotic host cells and produces this protein. Examples thereof are pMAL C2, pEF-BOS (Nucleic Acids Res. 18:5322 (1990) and so on), pME18S (Experimental Medicine: SUPPLEMENT, “Handbook of Genetic Engineering” (1992)), etc.

When bacteria, particularly E. coli, are used as host cells, an expression vector generally includes, at least, a promoter/operator region, an initiation codon, the DNA encoding the protein termination codon, terminator region, and replicon.

When yeast, animal cells, or insect cells are used as hosts, an expression vector preferably includes, at least, a promoter, an initiation codon, the DNA encoding the protein and a termination codon. It may also include the DNA encoding a signal peptide, enhancer sequence, 5′- and 3′-untranslated region of the gene encoding the protein, splicing junctions, polyadenylation site, selectable marker region, and replicon. The expression vector may also contain, if required, a gene for gene amplification (marker) that is usually used.

A promoter/operator region to express the protein in bacteria includes a promoter, an operator, and a Shine-Dalgarno (SD) sequence (e.g., AAGG). For example, when the host is Escherichia, it preferably includes Trp promoter, lac promoter, recA promoter, lambda PL promoter, b 1 pp promoter, tac promoter, or the like. Examples of a promoter to express the protein in yeast include PHO5 promoter, PGK promoter, GAP promoter, ADH promoter, and so on. When the host is Bacillus, examples include SLO1 promoter, SP02 promoter, penP promoter, and so on. When the host is a eukaryotic cell such as a mammalian cell, examples include SV40-derived promoter, retrovirus promoter, heat shock promoter, and so on, and preferably SV-40 and retrovirus-derived promoter. As a matter of course, the promoter is not limited to the above examples. In addition, using an enhancer is effective for expression.

A preferable initiation codon is, for example, a methionine codon (ATG).

A commonly used termination codon (e.g., TAG, TAA, TGA) is exemplified as a termination codon. Typically natural or synthetic terminators are used as a terminator region.

A “replicon” refers to a DNA capable of replicating the whole DNA sequence in host cells and includes a natural plasmid, an artificially modified plasmid (DNA fragment prepared from a natural plasmid), a synthetic plasmid, and so on. Examples of preferable plasmids are pBR322 or its artificial derivatives (DNA fragment obtained by treating pBR322 with appropriate restriction enzymes) for E. coli, yeast plasmid or yeast chromosomal DNA for yeast, and pRSVneo ATCC 37198, pSV2dhfr ATCC 37145, pdBPV-MMTneo ATCC 37224, pSV2neo ATCC 37149, and such for mammalian cells.

An enhancer sequence, polyadenylation site, and splicing junction typically used in the art, such as those derived from SV40, can also be used.

A typical selectable marker can be used according to known methods. Examples include resistance genes for antibiotics, such as tetracycline, ampicillin, or kanamycin.

Examples of genes for gene amplification include dihydrofolate reductase (DHFR) gene, thymidine kinase gene, neomycin resistance gene, glutamate synthase gene, adenosine deaminase gene, ornithine decarboxylase gene, hygromycin-B-phophotransferase gene, aspartate transcarbamylase gene, etc. Typically the genes described in the paragraph above are used for plasmid amplification in bacterial cells and the ones in this paragraph are used for selection of mammalian cells.

The expression vector used herein can be prepared by continuously and circularly linking at least the above-mentioned promoter, initiation codon, DNA encoding the protein, termination codon, and terminator region, to an appropriate replicon. If desired, appropriate DNA fragments (for example, linkers, restriction sites, and so on), can be used by known methods such as digestion with a restriction enzyme or ligation using T4 DNA ligase.

As used herein, “transformants” can be prepared by introducing the expression vector described above into host cells.

As used herein, “host” cells are not limited as long as they are compatible with an expression vector described above and can be transformed. Examples of host cells include various cells such as wild-type cells or artificially established recombinant cells usually used in the technical field (e.g., bacteria (e.g., Escherichia and Bacillus), yeast (e.g., Saccharomyces, Pichia, and such), animal cells, or insect cells).

Specific examples of suitable host cells include E. coli (e.g., DH5ₐₗₚₕₐ, TB1, HB101, and such), mouse-derived cells (e.g., COP, L, C127, Sp2/0, NS-1, NIH 3T3, and such), rat-derived cells (e.g., PC12, PC12h), hamster-derived cells (e.g., BHK, CHO, and such), monkey-derived cells (e.g., COS1, COS3, COS7, CV1, Velo, and such), and human-derived cells (e.g., Hela, diploid fibroblast-derived cells, myeloma cells, and HepG2, and such).

An expression vector can be introduced (transformed/transfected/transduced/electroporated) into host cells by known methods.

Transformation can be performed, for example, according to the method of Cohen et al. (Proc. Natl. Acad. Sci. USA, 69:2110 (1972)), protoplast method (Mol, Gen. Genet., 168:111 (1979)), or competent method (J. Mol. Biol., 56:209 (1971)) when the hosts are bacteria (E. coli, Bacillus subtilis, and such), the method of Hinnen et al. (Proc. Natl. Acad. Sci. USA, 75:1927 (1978)), or lithium method (J. Bacteriol., 153:163 (1983)) when the host is Saccharomyces cerevisiae, the method of Graham (Virology, 52:456 (1973)) when the hosts are animal cells, and the method of Summers et al. (Mol. Cell. Biol., 3:2156-2165 (1983)) when the hosts are insect cells.

The protein disclosed herein can be produced by cultivating transformants (in the present disclosure, this term includes transfectants) including an expression vector prepared as mentioned in nutrient media.

The nutrient media preferably include a carbon source, inorganic nitrogen source, or organic nitrogen source necessary for the growth of host cells (transformants). Examples of the carbon source include glucose, dextran, soluble starch, and sucrose; and examples of the inorganic or organic nitrogen source are ammonium salts, nitrates, amino acids, corn steep liquor, peptone, casein, meet extract, soy bean cake, and potato extract. If desired, they may comprise other nutrients (for example, an inorganic salt (for example, calcium chloride, sodium dihydrogenphosphate, and magnesium chloride), vitamins, antibiotics (for example, tetracycline, neomycin, ampicillin, kanamycin, and so on).

Cultivation of cell lines is performed by methods known in the art. Cultivation conditions such as temperature, pH of the media, and cultivation time are selected appropriately so that the protein is produced in large quantities.

Examples of isolation and purification methods include methods utilizing solubility, such as salting out and solvent precipitation method; a method utilizing the difference in molecular weight, such as dialysis, ultrafiltration, gel filtration, and sodium dodecyl sulfate-polyacrylamide gel electrophoresis; a method utilizing charges, such as ion exchange chromatography and hydroxylapatite chromatography; a method utilizing specific affinity, such as affinity column chromatography; a method utilizing the difference in hydrophobicity, such as reverse phase high performance liquid chromatography; and a method utilizing the difference in isoelectric point, such as isoelectric focusing.

By way of example, a polynucleotide sequence of the present disclosure may be identical to the reference sequence, that is be 100% identical, or it may include up to a certain integer number of nucleotide alterations as compared to the reference sequence. Such alterations are selected from the group including at least one nucleotide deletion, substitution, including transition and transversion, or insertion, and wherein said alterations may occur at the 5′ or 3′ terminus positions of the reference nucleotide sequence or anywhere between those terminus positions, interspersed either individually among the nucleotides in the reference sequence or in one or more contiguous groups within the reference sequence. The number of nucleotide alterations is determined by multiplying the total number of nucleotides in the reference nucleotide by the numerical percent of the respective percent identity (divided by 100) and subtracting that product from said total number of nucleotides in the reference nucleotide. Alterations of a polynucleotide sequence encoding the polypeptide may alter the polypeptide encoded by the polynucleotide following such alterations.

The term “codon” refers to a specific triplet of mononucleotides in the DNA chain or mRNA that make up an amino acid or termination signal.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (e.g., GAU and GAC triplets each encode Asp).

As used herein, the term “exogenous DNA” or “exogenous nucleic acid sequence” or “exogenous polynucleotide” refers to a nucleic acid sequence that was introduced into a cell or organelle from an external source. Typically, the introduced exogenous sequence is a recombinant sequence.

As used herein, the term “transfection” refers to the introduction of a nucleic acid sequence into the interior of a membrane enclosed space of a living cell, including introduction of the nucleic acid sequence into the cytosol of a cell as well as the interior space of a mitochondria, nucleus or chloroplast. The nucleic acid may be in the form of naked DNA or RNA, associated with various proteins, or the nucleic acid may be incorporated into a vector.

As used herein, the term “vector” or “expression vector” is used to denote a DNA molecule, linear or circular, which includes a segment encoding a polypeptide of interest operably linked to additional segments that provide for its transcription and translation upon introduction into a host cell or host cell organelles. Such additional segments include promoter and terminator sequences, and may also include one or more origins of replication, one or more selectable markers, an enhancer, a polyadenylation signal, etc. Expression vectors are generally derived from yeast or bacterial genome or plasmid DNA, animal virus genome, or viral DNA, or may contain elements of both.

“DNA regulatory sequences”, as used herein, are transcriptional and translational control sequences, such as promoters, enhancers, polyadenylation signals, termination signals, and the like, that provide for and/or regulate expression of a coding sequence in a host cell.

A “promoter sequence” is a DNA regulatory region in an operon capable of binding RNA polymerase in a cell and initiating transcription of a downstream (3′ direction) coding sequence. The promoter sequence is bound at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present disclosure.

The terms “chimeric”, “fusion” and “composite” are used to denote a protein, peptide domain or nucleotide sequence or molecule containing at least two component portions that are mutually heterologous in the sense that they are not, otherwise, found directly (covalently) linked in nature. More specifically, the component portions are not found in the same continuous polypeptide or gene in nature, at least not in the same order or orientation or with the same spacing present in the chimeric protein or composite domain. Such materials contain components derived from at least two different proteins or genes or from at least two non-adjacent portions of the same protein or gene. Composite proteins, and DNA sequences that encode them, are recombinant in the sense that they contain at least two constituent portions that are not otherwise found directly linked (covalently) together in nature.

The term “domain” in this context is not intended to be limited to a single discrete folding domain.

A “reporter polynucleotide” includes any gene that expresses a detectable gene product, which may be RNA or a reporter polypeptide. Reporter genes include coding sequences for which the transcriptional and/or translational products are readily detectable or selectable.

An “insertion” or “addition”, as used herein, refers to a change in an amino acid or nucleotide sequence resulting in the addition or insertion of one or more amino acid or nucleotide residues, respectively, as compared to the corresponding naturally occurring molecule.

A “deletion” or “subtraction”, as used herein, refers to a change in an amino acid or nucleotide sequence resulting in the deletion or subtraction of one or more amino acid or nucleotide residues, respectively, as compared to the corresponding naturally occurring molecule.

A “substitution”, as used herein, refers to the replacement of one or more amino acids or nucleotides by different amino acids or nucleotides, respectively.

A “mutation” is a heritable change in a DNA sequence relative to a reference “wild-type” DNA sequence. Mutations can occur as a result of a single base change, multiple base changes, or the addition or deletion of more than one nucleotide to a DNA sequence.

The term “genotoxicity” is used to broadly refer to any deleterious change in the genetic material of a cell, regardless of the mechanism by which the change is induced.

As used herein the term “mutagenicity” and “genotoxic activity” are used to refer to the ability of an agent (e.g., a chemical compound or a drug candidate) to cause a permanent change in the structure of the genetic material of a cell, which causes a heritable change in the effected cell. Contemplated changes include alterations in the sequences of the bases in the nucleic acid (gene mutation), structural changes to chromosomes (clastogenicity) and/or changes to the number of chromosomes present.

A “mutagen” or a “genotoxic agent” is an agent that creates or causes mutations. It is well-established that chemical mutagens vary in their modes of action. However, in general terms, a chemical mutagen changes a nucleic acid or nucleoside relative to the nucleotide sequence of a reference or “wild-type” genome. Generally speaking, a mutagen or genotoxic agent increases the frequency of reversion or forward mutation.

The term “mutant” is employed broadly to refer to a protein that differs in some way from a reference wild-type protein, where the protein may retain biological properties of the reference wild-type (e.g., naturally occurring) protein, or may have biological properties that differ from the reference wild-type protein. The term “biological property” of the subject proteins includes, but is not limited to, spectral properties, such as emission maximum, quantum yield, and brightness, and the like; in vivo and/or in vitro stability (e.g., half-life); and the like. Mutants can include single amino acid changes (point mutations), deletions of one or more amino acids (point-deletions), N-terminal truncations, C-terminal truncations, insertions, and the like. Mutants can be generated using standard techniques of molecular biology.

A “gene mutation” refers to a mutation that occurs entirely within one gene, or its upstream regulatory sequences and can comprise either a point mutation or other disruption of normal chromosomal structure that occurs entirely within one gene.

A “reversion assay” is an assay of genotoxic activity that detects a reverse mutation that confers normal function to a mutant gene thereby causing a gain of function. Typically, the genotoxic activity of compounds is evaluated using a bacterial reverse mutation assay that utilizes an amino acid-requiring (e.g., auxotrophic) tester strains of Salmonella typhimurium (S. typhimurium) or Escherichia coli (E. coli) to evaluate the genotoxic activity of a compound. Generally speaking, reversion assays are capable of detecting point mutations, such as a substitution, an addition or a deletion of one or more DNA bases, which are introduced into the genome of an affected tester strain.

A “forward mutation assay” is an assay of genotoxic activity, which detects “forward” mutations that alter a functional gene in a way that causes a loss, rather than a gain, of function.

A “wild-type” strain is capable of a full range of metabolic activities. For example, wild-type strains of Salmonella can synthesize all 20 amino acids from a single carbon source.

A “mutant” strain is not capable of all of the activities of the wild-type strain from which it is derived. For example, a mutant bacterial strain that is defective in its ability to synthesize the amino acid histidine (his strain) requires the presence of exogenous histidine in order to grow.

A “point mutation” is a change in one, or a small number of base pairs, in a DNA sequence. Point mutations may result from base pair substitutions or from small insertions or deletions.

A “transition” is a point mutation in which a purine is replaced with a purine or a pyrimidine is replaced with a pyrimidine.

A “transversion” is a point mutation in which a purine is replaced with a pyrimidine or a pyrimidine with a purine. Generally speaking, transitions are more common than tranversions because the former is not detected by the proofreading enzymes.

Alternatively, point mutation can also cause a nonsense mutation resulting from the insertion of a stop codon (amber, ochre, opal). Base pair mutations that generate a translation stop codon cause premature termination of translation of the coded protein.

A “frameshift mutation” results from the insertion or deletion of one or more nucleotides within a gene. The “reading frame” of a gene refers to the order of the bases with respect to the starting point for translation of the mRNA. Deletion of a single base pair results in moving ahead one base in all of the codons, and is often referred to as a positive frameshift. Addition of one base pair (or loss of two base pairs) shifts the reading frame behind by one base, and is often referred to as a negative frameshift.

As used herein the term “DNA Repair Mechanism” refers to any one of the potential repair mechanisms that exist in both prokaryotes and eukaryotes. For example: postreplication; mismatch repair; nucleotide excision-repair and photoreactivation or light-dependent repair (not found in mammals).

A “base pair substitution mutagen” is an agent that causes a base (e.g., nucleotide) change in DNA. In the context of a reversion test this change may occur at the site of the original mutation, or at a second site in the bacterial genome.

A “frameshift mutagen” is an agent that causes the addition or deletion of one or more base pairs in the DNA, thus changing the reading frame in the RNA.

As used herein, the term “hybridization” refers to the process of association of two nucleic acid strands to form an antiparallel duplex stabilized by means of hydrogen bonding between residues of the opposite nucleic acid strands.

“Hybridizing” and “binding”, with respect to polynucleotides, are used interchangeably. The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

By “administration” is meant introducing an embodiment of the present disclosure into a subject. Administration can include routes, such as, but not limited to, intravenous, oral, topical, subcutaneous, intraperitoneal, intraarterial, inhalation, vaginal, rectal, nasal, introduction into the cerebrospinal fluid, or instillation into body compartments can be used.

In accordance with the present disclosure, “a detectably effective amount” of embodiments of the present disclosure is defined as an amount sufficient to yield an acceptable image using equipment that is available for clinical use. A detectably effective amount of the embodiments of the present disclosure may be administered in more than one injection. The detectably effective amount of embodiments of the present disclosure can vary according to factors such as the degree of susceptibility of the individual, the age, sex, and weight of the individual, idiosyncratic responses of the individual, the dosimetry, and the like. Detectably effective amounts of embodiments of the present disclosure can also vary according to instrument and film-related factors. Optimization of such factors is well within the level of skill in the art.

As used herein, the term “organelle” refers to cellular membrane-bound structures such as the chloroplast, mitochondrion, and nucleus. The term “organelle” includes natural and synthetic organelles.

As used herein, the term “non-nuclear organelle” refers to any cellular membrane bound structure present in a cell, except the nucleus.

As used herein, the term “host” or “organism” includes humans, mammals (e.g., cats, dogs, horses, etc.), living cells, and other living organisms. In particular, the host is a human subject. A living organism can be as simple as, for example, a single eukaryotic cell or as complex as a mammal. Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications.

The term “link” as used herein refers to a physical linkage as well as linkage that occurs by virtue of co-existence within a biological particle, e.g., phage, bacteria, yeast or other eukaryotic cell.

The construction of expression vectors and the expression of genes in transfected cells involves the use of molecular cloning techniques also well known in the art. Sambrook et al., Molecular Cloning—A Laboratory Manual, Cold Spring Harbor Laboratory, Cold Spring Harbor, N.Y., (1989) and Current Protocols in Molecular Biology, F. M. Ausubel et al., eds., (Current Protocols, a joint venture between Greene Publishing Associates, Inc. and John Wiley & Sons, Inc., most recent Supplement).

Nucleic acids used to transfect cells with sequences coding for expression of the polypeptide of interest generally will be in the form of an expression vector including expression control sequences operatively linked to a nucleotide sequence coding for expression of the polypeptide. As used, the term “nucleotide sequence coding for expression of” a polypeptide refers to a sequence that, upon transcription and translation of mRNA, produces the polypeptide. This can include sequences containing, e.g., introns. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (e.g., ATG) in front of a protein-encoding gene, splicing signals for introns, maintenance of the correct reading frame of that gene to permit proper translation of the mRNA, and stop codons.

Methods that are well known to those skilled in the art can be used to construct expression vectors containing the fluorescent indicator coding sequence and appropriate transcriptional/translational control signals. These methods include in vitro recombinant DNA techniques, synthetic techniques and in vivo recombination/genetic recombination. (See, for example, the techniques described in Maniatis, et al., Molecular Cloning A Laboratory Manual, Cold Spring Harbor Laboratory, N.Y., 1989).

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl method by procedures well known in the art. Alternatively, MgCl or RbCl can be used. Transformation can also be performed after forming a protoplast of the host cell or by electroporation.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be cotransfected with DNA sequences encoding the calcium sensing system of the present disclosure, and a second foreign DNA molecule encoding a selectable phenotype, such as the herpes simplex virus thymidine kinase gene. Another method is to use a eukaryotic viral vector, such as simian virus 40 (SV40) or bovine papilloma virus, to transiently infect or transform eukaryotic cells and express the protein. (Eukaryotic Viral Vectors, Cold Spring Harbor Laboratory, Gluzman ed., 1982). Preferably, a eukaryotic host is utilized as the host cell as described herein.

Techniques for the isolation and purification of either microbially or eukaryotically expressed polypeptides of the embodiments of the present disclosure may be by any conventional means such as, for example, preparative chromatographic separations and immunological separations such as those involving the use of monoclonal or polyclonal antibodies or antigen.

A variety of host-expression vector systems may be utilized to express embodiments of the present disclosure. These include, but are not limited to, microorganisms such as bacteria transformed with recombinant bacteriophage DNA, plasmid DNA or cosmid DNA expression vectors; yeast transformed with recombinant yeast expression vectors containing the calcium sensing system sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid vectors containing the calcium sensing system sequences; insect cell systems infected with recombinant virus expression vectors (e.g., baculovirus) vectors; or animal cell systems infected with recombinant virus expression vectors (e.g., retroviruses, adenovirus, vaccinia virus vectors containing, or transformed animal cell systems engineered for stable expression.

Depending on the host/vector system utilized, any of a number of suitable transcription and translation elements, including constitutive and inducible promoters, transcription enhancer elements, transcription terminators, etc. may be used in the expression vector (See, e.g., Bitter, et al., Methods in Enzymology 153:516-544, 1987). For example, when cloning in bacterial systems, inducible promoters such as pL of bacteriophage, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the inserted fluorescent indicator coding sequence.

In bacterial systems a number of expression vectors may be advantageously selected depending upon the use intended for calcium sensing system.

In yeast, a number of vectors containing constitutive or inducible promoters may be used. For a review see, Current Protocols in Molecular Biology, Vol. 2, Ed. Ausubel, et al., Greene Publish. Assoc. & Wiley Interscience, Ch. 13, 1988; Grant, et al., Expression and Secretion Vectors for Yeast, in Methods in Enzymology, Eds. Wu & Grossman, 31987, Acad. Press, N.Y., Vol. 153, pp. 516-544, 1987; Glover, DNA Cloning, Vol. II, IRL Press, Wash., D.C., Ch. 3, 1986; and Bitter, Heterologous Gene Expression in Yeast, Methods in Enzymology, Eds. Berger & Kimmel, Acad. Press, N.Y., Vol. 152, pp. 673-684, 1987; and The Molecular Biology of the Yeast Saccharomyces, Eds. Strathern et al., Cold Spring Harbor Press, Vols. I and II, 1982. A constitutive yeast promoter such as ADH or LEU2 or an inducible promoter such as GAL may be used (Cloning in Yeast, Ch. 3, R. Rothstein In: DNA Cloning Vol. 11, A Practical Approach, Ed. DM Glover, IRL Press, Wash., D.C., 1986). Alternatively, vectors may be used which promote integration of foreign DNA sequences into the yeast chromosome.

In cases where plant expression vectors are used, the mutation assay system may be driven by any of a number of promoters. For example, viral promoters such as the 35S RNA and 19S RNA promoters of CaMV (Brisson, et al., Nature 310:511-514, 1984), or the coat protein promoter to TMV (Takamatsu, et al., EMBO J. 6:307-311, 1987) may be used; alternatively, plant promoters such as the small subunit of RUBISCO (Coruzzi, et al., 1984, EMBO J. 3:1671-1680; Broglie, et al., Science 224:838-843, 1984); or heat shock promoters, e.g., soybean hsp17.5-E or hsp17.3-B (Gurley, et al., Mol. Cell. Biol. 6:559-565, 1986) may be used. These constructs can be introduced into plant cells using Ti plasmids, Ri plasmids, plant virus vectors, direct DNA transformation, microinjection, electroporation, etc. For reviews of such techniques see, for example, Weissbach & Weissbach, Methods for Plant Molecular Biology, Academic Press, N.Y., Section VIII, pp. 421-463, 1988; and Grierson & Corey, Plant Molecular Biology, 2d Ed., Blackie, London, Ch. 7-9, 1988.

An alternative expression system, which could be used to express mutation assay system, is an insect system. In one such system, Autographa californica nuclear polyhedrosis virus (AcNPV) is used as a vector to express foreign genes. The virus grows in Spodoptera frugiperda cells. The calcium sensing system sequences may be cloned into non-essential regions (for example, the polyhedrin gene) of the virus and placed under control of an AcNPV promoter (for example the polyhedrin promoter). Successful insertion of the calcium sensing system sequences will result in inactivation of the polyhedrin gene and production of non-occluded recombinant virus (e.g., virus lacking the proteinaceous coat coded for by the polyhedrin gene). These recombinant viruses are then used to infect Spodoptera frugiperda cells in which the inserted gene is expressed, see Smith, et al., J. Viol. 46:584, 1983; Smith, U.S. Pat. No. 4,215,051.

DNA sequences encoding the mutation assay system of the present disclosure can be expressed in vitro by DNA transfer into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, in other words when the foreign DNA is continuously maintained in the host, are known in the art.

“Physical linkage” refers to any method known in the art for functionally connecting two molecules (which are termed “physically linked”), including without limitation, recombinant fusion with or without intervening domains, intein-mediated fusion, non-covalent association, covalent bonding (e.g., disulfide bonding and other covalent bonding), hydrogen bonding; electrostatic bonding; and conformational bonding, e.g., antibody-antigen, and biotin-avidin associations.

As used herein, “linker” or “spacer” refers to a molecule or group of molecules that connects two molecules, such as a fluorescent binding ligand and a display protein or nucleic acid, and serves to place the two molecules in a preferred configuration.

The term “autologous” referred to here indicates that the cell donor was the same as the recipient; whereas the term “allogeneic” means that the cell donor is someone other than the recipient.

“Transformed” means a cell into which (or into an ancestor of which) has been introduced, by means of recombinant nucleic acid techniques, a heterologous nucleic acid molecule. “Heterologous” refers to a nucleic acid sequence that either originates from another species or is modified from either its original form or the form primarily expressed in the cell.

“Transgene” means any piece of DNA, which is inserted by artifice into a cell, and becomes part of the genome of the organism (e.g., either stably integrated or as a stable extrachromosomal element) which develops from that cell. Such a transgene may include a gene which is partly or entirely heterologous (e.g., foreign) to the transgenic organism, or may represent a gene homologous to an endogenous gene of the organism. Included within this definition is a transgene created by the providing of an RNA sequence that is transcribed into DNA and then incorporated into the genome. The transgenes used herein include DNA sequences that encode the fluorescent indicator that may be expressed in a transgenic non-human animal. The term “transgenic” as used herein additionally includes any organism whose genome has been altered by in vitro manipulation of the early embryo or fertilized egg or by any transgenic technology to induce a specific gene knockout. The term “gene knockout” as used herein, refers to the targeted disruption of a gene in vivo with complete loss of function that has been achieved by any transgenic technology familiar to those in the art. As used herein, the term “transgenic” includes any transgenic technology familiar to those in the art which can produce an organism carrying an introduced transgene or one in which an endogenous gene has been rendered non-functional or “knocked out.”

Another method of transfection includes nucleofection which is an efficient and reproducible method to transfer of polynucleotides into cells. The technology is an improved version of electroporation. The cells of interest can transfected by nucleofection using the Nucleofector™ system from Amaxa GmbH (Cologne, Germany). The Nucleofector™ technology is a highly efficient non-viral gene transfer method. Cell-type specific combinations of electrical current and solutions increase the capacity to transfer polyanionic macromolecules directly into the nucleus. Thus, cells with limited potential to divide, like many primary cells, become accessible for efficient gene transfer. Conditions for each cell type were optimized using manufactures' guidelines.

An “imaging reporter gene” is here defined as any gene that can encode a protein that can be detected in a living subject using an imaging modality. The protein product of an imaging reporter gene may itself emit a signal that is detectable by an imaging system; otherwise it can be detected using a signal emitting probe (“imaging reporter probe”). The protein product of the imaging reporter gene may emit fluorescence upon excitation by a specific range of wavelengths (“optical reporter gene”). The imaging reporter probe may emit positrons; hence would be detectable by positron emission tomography (PET). The imaging reporter probe may emit gamma radiation; hence it would be detectable by a gamma camera or single photon emission computed tomography (SPECT). The imaging reporter probe may be detectable by magnetic resonance imaging. The imaging reporter probe may emit bioluminescence upon interaction with the protein product of the reporter gene (“optical imaging probe”). In some instances the terms “nuclear imaging reporter gene” or “MRI based reporter gene” and “nuclear imaging reporter probe” or “MRI based reporter probe” are used.

As used herein, the terms “treatment”, “treating”, and “treat” are defined as acting upon a disease, disorder, or condition with an agent to reduce or ameliorate the pharmacologic and/or physiologic effects of the disease, disorder, or condition and/or its symptoms. “Treatment,” as used herein, covers any treatment of a disease in a host (e.g., a mammal, typically a human or non-human animal of veterinary interest), and includes: (a) reducing the risk of occurrence of the disease in a subject determined to be predisposed to the disease but not yet diagnosed as infected with the disease (b) impeding the development of the disease, and (c) relieving the disease, e.g., causing regression of the disease and/or relieving one or more disease symptoms. “Treatment” is also meant to encompass delivery of an inhibiting agent to provide a pharmacologic effect, even in the absence of a disease or condition. For example, “treatment” encompasses delivery of a disease or pathogen inhibiting agent that provides for enhanced or desirable effects in the subject (e.g., reduction of pathogen load, reduction of disease symptoms, etc.).

As used herein, the terms “prophylactically treat” or “prophylactically treating” refers completely or partially preventing a disease or symptom thereof and/or may be therapeutic in terms of a partial or complete cure for a disease and/or adverse effect attributable to the disease.

### General Discussion

The present disclosure includes engineered cells, methods of imaging specific engineered cells, imaging reporter gene/probe systems, kits, and the like. Embodiments of the present disclosure can be used to image, detect (and visualize), quantitate, study, monitor, evaluate, and/or screen, biological events in vivo or in vitro, such as, but not limited to, trafficking of cells to a target such as precancerous tissue, cancer, tumors, and related biological events, as well as other diseases or conditions.

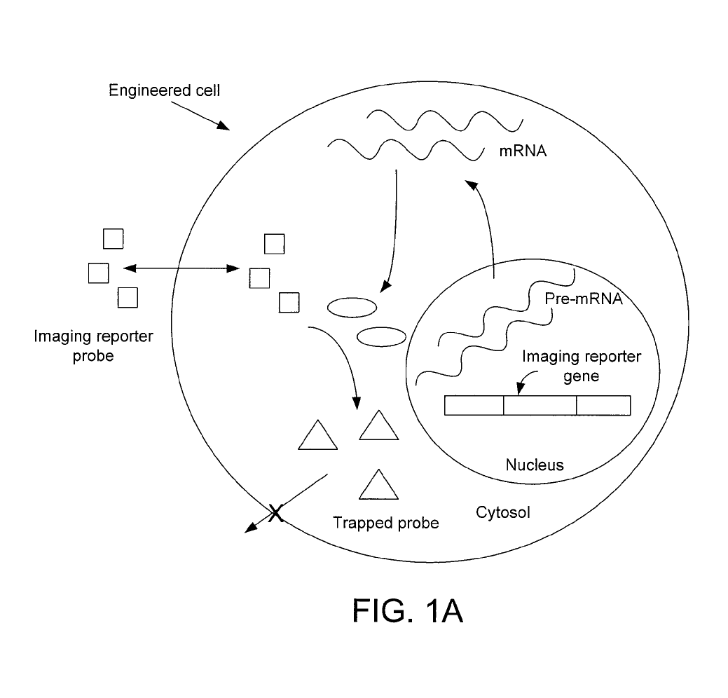

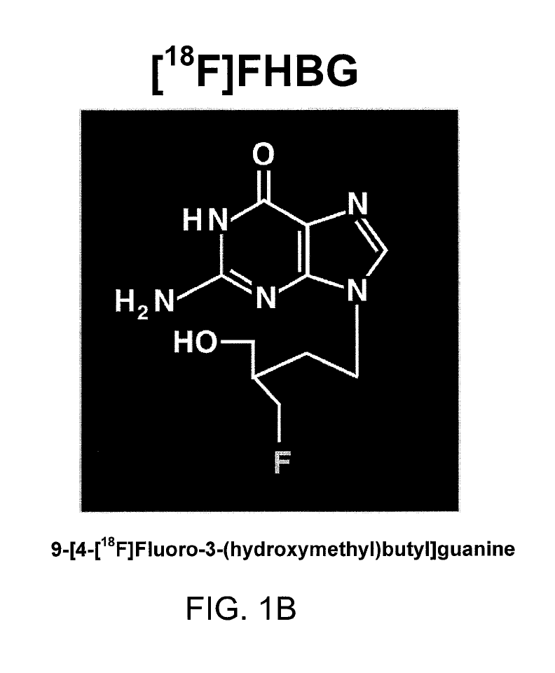

In general and as shown in FIG. 1A, embodiments of the present disclosure include methods of imaging engineered cells introduced into a human subject. In an embodiment, the engineered cells can be introduced as imaging agents, therapeutic agents, or as potential therapeutic agents. The engineered cells are genetically engineered ex vivo (outside the body of the human subject) to express an imaging reporter gene. In an embodiment, the imaging reporter gene is expressed in the engineered cell to generate an imaging reporter gene product. In an embodiment, the administered engineered cells can have a specific affinity for a particular tissue, tumor, cells, or the like, or may have been designed with the intention of a specific target, but may not necessarily have a target.

Subsequent and/or prior to the introduction of the engineered cells to the human subject, an imaging reporter probe is introduced to the human subject. In an embodiment, the imaging reporter gene product (e.g., an enzyme) can interact (e.g., react with and phosphorylate) with the imaging reporter probe to produce a modified imaging reporter probe (shown as “trapped probe” in FIG. 1). In an embodiment, the modified imaging reporter probe accumulates in the engineered cells (e.g., engineered therapeutic cells) that have been delivered to the human subject. The human subject or an area of the human subject can be imaged using a nuclear imaging system. Thus, the accumulated modified imaging reporter probe can be imaged. In this regard, embodiments of the present disclosure can be used to image (indirectly) the location and quantity of live engineered cells. In another embodiment, an imaging reported probe is not administered to the subject or is not used, and the imaging reporter gene product can accumulate in the cell and can be imaged. In addition, the imaging reporter gene and/or imaging reporter gene product can interact with compounds already present in the subject, and those compounds (the compound or a product of the compound) can accumulate and can be imaged.

In an embodiment, genetically engineered cytolytic T cells (CTL) were delivered (e.g., administered) to a human subject. The CTLs had an affinity for glioma tumor cells. The CTLs expressed an imaging reporter gene. Then, an imaging reporter probe was administered to the human subject. The imaging reporter probe entered the CTL and was modified (e.g., chemically altered) so that it could no longer exit the CTL. Subsequently, the human subject or an area of the human subject is non-invasively imaged using a nuclear imaging system to detect for the presence of the modified imaging reporter probe that has accumulated in the CTL. In this regard, the modified imaging reporter probe reveals the location of the CTLs, which had an affinity for the glioma tumor target, thereby imaging the glioma tumor. The nuclear imaging system used was PET, because the probe was a positron emitter.

In another embodiment, other reporter genes can be used, such that the administered cells would have been detectable by SPECT, fluorescence, bioluminescence, or MR imaging, or other imaging technology, as well as PET. In an embodiment, an imaging reporter probe is not used.

The term “cancer”, as used herein, shall be given its ordinary meaning, as a general term for diseases in which abnormal cells divide without control. Cancer cells can invade nearby tissues and can spread through the bloodstream and lymphatic system to other parts of the body.

Gliomas encompass a group of neoplasms that represents the most common type of primary CNS tumors. They are all characterized by including at least a subset of neoplastic cells that phenotypically resemble macroglial cells. As defined by the World Health Organization, they vary markedly in aggressiveness, occurring in forms that are curable if resected (Grade 1 gliomas) to those which are fatal within a year (Grade IV or glioblastoma). Although they do not commonly metastasize or spread outside the central nervous system, glioma cells commonly metastasize within this tissue, a property that renders them extremely difficult to treat.

As mentioned above, embodiments of the present disclosure include an imaging reporter gene/probe system and imaging reporter gene/probe based cells. The operation of the imaging reporter gene/probe system and imaging reporter gene/probe based cells are described herein.

In an embodiment, the engineered is administered to a host. Although the application primarily refers to a human, embodiments of the present disclosure can be used in a host.

### Engineered Cells and Imaging Reporter Gene

The engineered cells (also referred to as “imaging reporter gene/probe based cell”) can include, but are not limited to, allogeneic or autologous human immune cells (e.g., lymphocytes; antigen presenting cells (e.g. dendritic cells); neutrophils; granulocytes; macrophages; mast cells), human cell lines, human stem cells (e.g. embryonic; adult; mesenchymal), and the like. In an embodiment, the engineered cell is a cytotoxic T cell (CTL). In an embodiment, the engineered cell is an autologous cell. In an embodiment, the engineered cell is an autologous CTL. The term “autologous” referred to here indicates that the cell donor was the same as the recipient; whereas the term “allogeneic” means that the cell donor is someone other than the recipient.

In an embodiment, the engineered cell of the present disclosure can have a specific affinity for a target. In this regard, the term “affinity” means that the engineered cells are preferentially attracted to the target(s) as opposed to all other targets in the human subject. The engineered cells can be engineered to have such affinity by using one or more polypeptides (e.g., proteins) or chemical moieties to provide the affinity for the target.

In an embodiment, the engineered cell is a CTL that has an affinity for a target. In an embodiment, the CTLs express a protein receptor called IL-13 Zetakine that specifically targets the CTLs to cells in malignant glioma tumors. Additional embodiments of the CTL can be engineered to have an affinity for one or more cancers, pre-cancerous cells, tumors, or cells or tissue associated with other diseases or conditions. In an embodiment, the CTLs can be selected from Cytolytic CD8+ T cells of the same patient who subsequently receives the genetically engineered CTLs.

As mentioned above, the engineered cell can be genetically engineered ex vivo to express an imaging reporter gene. In an embodiment, the engineered cell includes an imaging reporter gene not naturally present in the human subject. Although, some reporter genes have been derived from endogenous mammalian genes and they may also be used to image cells in humans. In an embodiment, the imaging reporter gene is expressed in the engineered cells to generate an imaging reporter gene product. In an embodiment, the imaging reporter gene product (e.g., an enzyme) can interact (e.g., react with and phosphorylate) with the imaging reporter probe to produce a modified imaging reporter probe.

The imaging reporter gene of the present disclosure can include a reporter gene that will be detectable (directly (e.g., detecting the imaging reporter gene product) or indirectly (e.g., using an imaging reporter probe)) within cells inside a living human. In an embodiment, the imaging reporter gene is Herpes Simplex Virus 1 thymidine kinase (HSV1-tk) positron emission tomography (PET) reporter gene (PRG) (Accession Numbers EU814922, EU541360-EU541370, SEQ ID NO: 1). HSV1-tk encodes the enzyme protein (HSV1-TK) (SEQ ID NO: 2), which can phosphorylates some nucleotide analogs far better than a mammalian TK enzyme. Phosphorylation of the imaging reporter probe results in entrapment of the imaging reporter probe only within cells expressing HSV1-tk (See FIG. 1A).

In an embodiment, the imaging reporter gene can include a mutant of Herpes Simplex Virus 1 thymidine kinase (e.g., HSV1-sr39tk (SEQ ID NO: 3), HSV1-A167Ytk, and HSV1-A167Ysr39tk) that phosphorylate [¹⁸F]FHBG and traps it within reporter gene expressing cells. In an embodiment, imaging reporter probes for HSV1-tk or its mutants can include a radiolabeled thymidine or acycloguanosine analog.

In an embodiment, the imaging reporter gene can include other PET or SPECT reporter genes such as, but not limited to, Dopamine 2 Receptor (Accession Numbers NM 000795 and NM 016574) or Sodium Iodide Symporter (Accession # NM 000453) and specific radionuclide probes (e.g., [¹⁸F]FESP) for the reporter genes.

In an embodiment, the imaging reporter gene can include an MRI reporter gene (e.g., MagA, Accession # YP423353 and the artificial CEST agent which encodes for 200 lysine residues) that may or may not require a probe for imaging. Many MRI reporter genes cause accumulation of iron inside the cell expressing them, generating T2 weighed MRI contrast. The probeless CEST agent generates MRI contrast through proton exchange.

In an embodiment, the imaging reporter gene can include an optical reporter gene (e.g., bioluminescence Rluc8, Accession # EF446136 and fluorescence DSred, Accession # FJ226078) that may or may not require an imaging reporter probe. Many of the fluorescent reporter genes generate light upon excitation by light of a different wavelength than their emission light wavelength, without requiring a reporter probe.

In an embodiment, the imaging reporter transgene can be delivered into the cells using non-viral and viral vectors as well as by using any other transgene delivery techniques including electroporation and nucleofection. Such methods are known to those of skill in the art and are described briefly above. In an embodiment, the HSV1-tk reporter and Hygromycin selection transgenes, regulated by the cytomegalovirus promoter, were electroporated into autologous patient CTLs. The Hygromycin resistant CTLs were then selected to generate CTL clones that were then expanded to a count of 1×10¹⁰ cells in culture before being infused into the patient. In general, this procedure has been described by Kahlon et al. in (2004) Cancer Res. 64, 9160-9166, which is incorporated herein by reference for the corresponding discussion.

The engineered cell may be administered intravenously, intraperitoneally, subcutaneously, into the cerebrospinal fluid, or directly injected/implanted into a specific organ or tissue within the body of a human. In an embodiment, the CTLs were delivered or administered into the patient by injecting them into the recurrent glioma tumor resection site.

### Imaging Reporter Probe

The imaging reporter probe of the present disclosure freely enters and exits the CTL. In an embodiment, the imaging reporter gene product (e.g., an enzyme protein) interacts with the imaging reporter probe to produce a modified imaging reporter probe (e.g., a phosphorylated imaging reporter probe). The modified imaging reporter probe accumulates in the CTL because the modified imaging reporter probe can not exit the CTL. In an embodiment, the imaging reporter probe interacts with the imaging reporter gene product and accumulates in the CTL.

It should also be noted that certain embodiments of the present disclosure do not need an imaging reporter probe administered to the subject. In this regard, the imaging reporter gene and/or the imaging reporter gene product interacts with compounds (e.g., iron present in the body), and the compound (or a metal derivative or the like of the compound) accumulates in the CTL.

The imaging reporter probe can include imaging probes that are specific for a clinically useful imaging reporter gene that can be used to label cells for imaging in living humans. In an embodiment, the imaging reporter probe can include [¹⁸F]FHBG, [¹⁸F]FEAU, or [¹²⁴I]FIAU. In addition, the imaging reporter probe can include SPECT or PET radionuclide labeled tracers that can be specifically phosphorylated by HSV1-TK enzyme or its mutants (e.g., HSV1-sr39TK). In an embodiment, the imaging reporter probe can include PET or SPECT radionuclide labeled tracers that are specific for other PET or SPECT reporter genes (e.g., [¹⁸F]FESP that can specifically bind D₂R PET reporter gene). In an embodiment, the imaging reporter probe can include MRI contrast agents that only accumulate in cells expressing their respective MRI reporter gene.

In an embodiment, the imaging reporter probe is an imaging reporter probe that has the characteristic of being able to detect the expression of HSV1-tk or HSV1-sr39tk PET reporter genes in cells within humans.

In an embodiment, the imaging reporter probe can be imaged using PET. In an exemplary embodiment the imaging reporter probe is 9-[4-[¹⁸F]fluoro-3-(hydroxymethyl)butyl]guanine ([¹⁸F]FHBG)m (See FIG. 1B). [¹⁸F]FHBG can be phosphorylated to form a phosphorylated [¹⁸F]FHBG, which can be detected by PET.

The imaging reporter probes are usually administered intravenously. In an embodiment, [¹⁸F]FHBG was administered intravenously (See Example 1). However, other methods of administration may also be used.

[¹⁸F]FHBG can be synthesized according to a protocol described by Yaghoubi et al. ((2001) J Nucl Med 42, 1225-1234, which is incorporated herein by reference for the corresponding discussion). [¹⁸F]FHBG has been evaluated for pharmacokinetics, dosimetry and safety in humans (2) and safety in rats and rabbits ((2006) J Nucl Med 47, 706-715, which is incorporated herein by reference for the corresponding discussion). The protocol for imaging humans with [¹⁸F]FHBG has been written in detail by Yaghoubi et al. ((2007) Nat Protoc 1, 3069-3075, which is incorporated herein by reference for the corresponding discussion).

### Methods of Use

Embodiments of this disclosure include, but are not limited to: methods of imaging cells, genetically engineered ex vivo to express an imaging reporter gene, in a human subject.

In general, the engineered cells and/or the imaging reporter gene/probe system of the present disclosure can be used to image the localization and quantity of living cells genetically engineered to express an imaging reporter gene in living subjects (e.g., a living human). Embodiments of this disclosure include reporter gene/probe based imaging of cells in humans. For example, CTLs have been genetically engineered to express the HSV1-tk imaging reporter gene. Then the CTLs were injected into the surgically resected tumor site of a patient's brain. [¹⁸F]FHBG was injected intravenously to image the location of living CTLs following their administration. [¹⁸F]FHBG was phosphorylated by the product of the imaging reporter gene. The phosphorylated [¹⁸F]FHBG accumulated in CTLs, which corresponded to the site of injection as well as a glioma tumor grown at a remote site of the patient's brain.

In an embodiment, cells that can be administered into a human can be genetically engineered consistent with embodiments of the present disclosure to include a clinically relevant imaging reporter gene. Thus, embodiments of the present disclosure enable the location, survival, quantity, and status of such genetically engineered cells to be imaged in a living human subject.

### Kits

In embodiment, the disclosure encompasses kits that include engineered cells and directions for use. In another embodiment, the disclosure encompasses kits that include engineered cells and an imaging reporter probe, and directions for use. As described herein, the engineered cells and/or the imaging reporter probe can be administered to a human subject. In addition, the kit can include one or more devices to administer the one or more of the components of the present disclosure. In particular, the device can include syringes and the like.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

### EXAMPLES

Now having described the embodiments of the disclosure, in general, the example describes some additional embodiments. While embodiments of present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### Introduction

A 57 years old caucasian male had been diagnosed with grade IV glioblastoma multiforme (GBM). The patient was enrolled in an FDA authorized (BB-IND 10109) adoptive cellular gene immunotherapy (ACGT) trial at City of Hope National Medical Center (COHNMC IRB#01020, See Inclusion and Exclusion Criteria noted below). Leukapheresis was initiated after obtaining informed consent and following completion of the primary therapy. The leukapheresis product was transferred to COHNMC's T cell production facility to initiate T cell cultures.

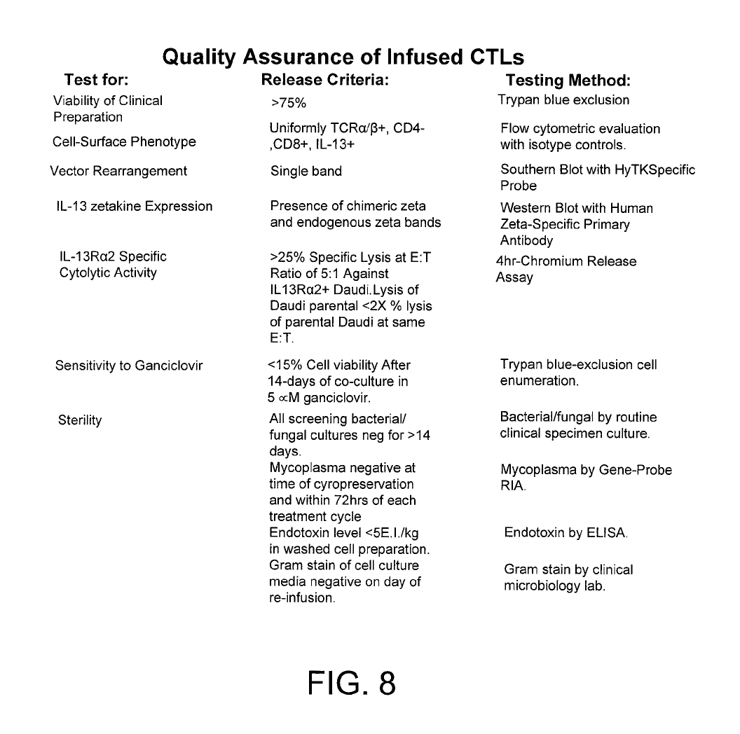

Nine months after initial diagnosis of GBM a recurrent tumor adjacent to the resection cavity was detected by MRI. The recurrent tumor was resected and a Rickham reservoir was inserted to allow infusion of genetically engineered autologous CD8⁺ cytolytic T cells (CTL). T cells were isolated from the patient's peripheral blood mononuclear cells and electroporated, delivering a plasmid DNA construct encoding IL-13 zetakine and Hygromycin/Herpes Simplex virus 1 thymidine kinase (HSV1-tk) genes under the transcriptional control of a modified human Elongation Factor-1α (EF-1α) promoter and the cytomegalovirus (CMV) immediate/early promoter, respectively in a cell production facility at COHNMC. Hygromycin resistant CTLs were cloned in limiting dilution than expanded using the REM method to numbers in excess of 10⁹ and cryopreserved. Following diagnosis of relapse, cryopreserved cells were thawed, expanded and formulated for intracranial infusion in 2 cc of preservative-free normal saline (PFNS). These cells were infused over a period of 5 weeks on Mondays, Wednesdays and Fridays, with a break on week 3. The patient started with a cell dose of 1×10⁷. Since he tolerated that dose well, his cell infusion increased to 1×10⁸ per day. By the end of the CTL infusions the patient had received approximately 1×10⁹ genetically engineered autologous CTLs (Refer to the quality assurance analysis of infused CTLs, FIG. 8). During the initial course of therapy, an enhancing lesion evolved in the posterior corpus callosum in the contralateral hemisphere. This lesion was biopsy proven GBM, and the patient received additional focal radiation therapy, avastin, and BCNU. Upon further progression the patient received a series of intralesional T cell doses. 14 weeks thereafter MRI revealed a major tumor regression. The patient survived 14-months from the time of initial recurrence. During the T-cell therapy no serious unexpected adverse events were encountered and the major complaint was expected intermittent headache.

Three days after completion of 5-week CTL infusions the patient had an investigational positron emission tomography (PET) scan to detect the CTLs within his body. The CTLs were imaged with the PET reporter probe 9-[4-[¹⁸F]Fluoro-3-(hydroxymethyl)butyl]guanine ([¹⁸F]FHBG), because they constitutively express the PET reporter gene (PRG) HSV1-tk. (4) [¹⁸F]FHBG is approved by the FDA as an investigational new drug (IND #61,880) for PET imaging at UCLA and Stanford University nuclear medicine clinics. UCLA's medical internal review board (M-IRB) has approved [¹⁸F]FHBG PET imaging in normal volunteers, glioma patients and glioma patients who are enrolled in adoptive cellular gene therapy, when the infused cells express the PRG HSV1-tk. Stanford University's M-IRB has approved [¹⁸F]FHBG PET imaging in glioma patients. COHNMC's M-IRB has approved referral of the patient's enrolled in the CTL therapy study for [¹⁸F]FHBG PET imaging at UCLA.

The patient gave informed consent and came to UCLA Nuclear Medicine clinic, where he was first administered a mini-mental status exam (MMSE) and a urine sample was collected for baseline urine-analysis. Two intravenous (iv) lines were inserted, one into each arm, from one of which was collected 2 ml blood for baseline CBC and 5 ml blood for baseline chemistry analysis. The patient's baseline vital signs, including temperature, heart rate, blood pressure, blood oxygen %, respiratory rate and electrocardiogram were recorded. With the exception of EKG, which was recorded at approximately every 15 minutes (up to 2-hours) after [¹⁸F]FHBG injection, the other vital signs were recorded at approximately 5, 10, 15, 30, 60, and 120 minutes after [¹⁸F]FHBG injection. All of these vital signs were again measured and recorded the day after and one week after the imaging session. Furthermore, blood and urine was collected on those follow-up days for laboratory tests. Finally, another MMSE was taken a week after [¹⁸F]FHBG injection to rule out any effect on cognitive functions. The patient's MMSE score was 25 at both baseline and follow-up.

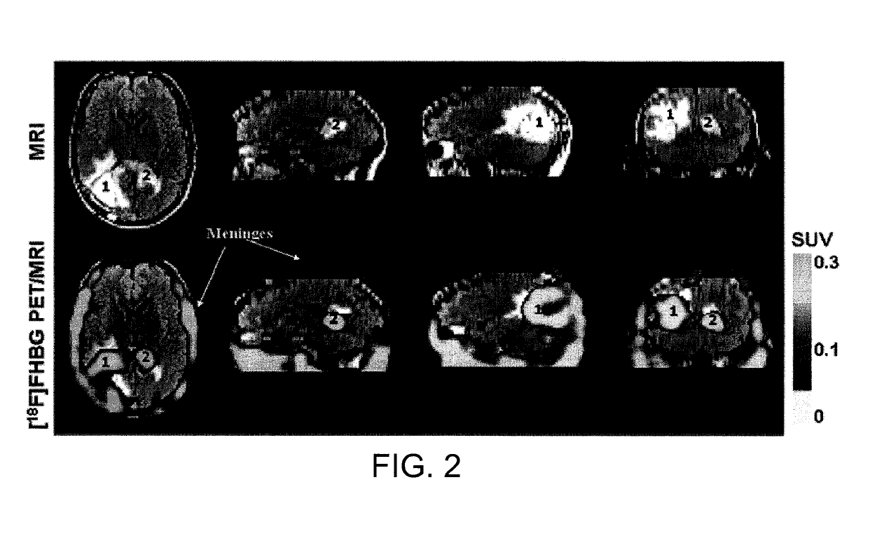

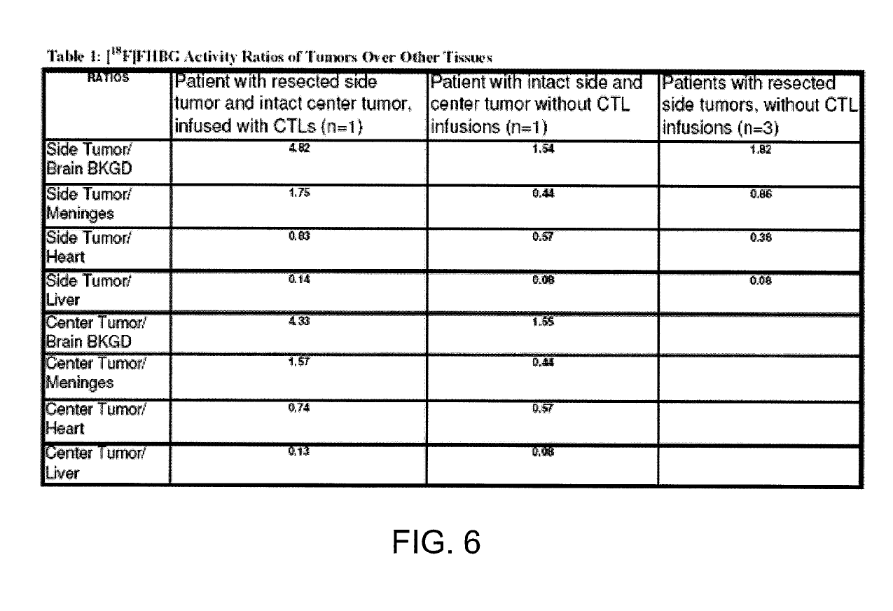

254.62 MBq (6.88 mCi) [¹⁸F]FHBG was injected through the other iv line (FDA limit 7 mCi). At approximately two-hours and fifteen minutes after [¹⁸F]FHBG injection, the whole-body PET emission scan started from the patient's head to bottom, using an ECAT EXACT HR+PET scanner (CTI/Siemens, Inc., Knoxville, Tenn.). The whole-body scan consisted of 7 bed positions (7-minutes each). Three minute transmission scans followed each 7 minute emission scans. A two bed position head scan immediately followed completion of the whole-body scan. FIG. 2 shows enhanced [¹⁸F]FHBG accumulation within the tumor resection site, where CTLs had been infused. In addition, enhanced [¹⁸F]FHBG accumulation was observed near the patient's corpus callosum. This indicates trafficking of infused CTLs to the remote corpus callosum tumor. The level of [¹⁸F]FHBG accumulation is greater than what has been observed in control GBM patients who had tumor resections or intact tumors. Table 1 (FIG. 6) includes quantitative data of tumor to background ratios, comparing [¹⁸F]FHBG accumulation levels of the CTL infused patient described in this example and control patients. Relative to similar controls, [¹⁸F]FHBG accumulation is 2.6× higher in the patient's tumor resection site and 2.8× higher in the remote corpus callosum tumor site. Cell culture uptake assays of the patient's CTLs later confirmed their HSV1-tk expression, consistent with previous in vitro assays confirming CTL's susceptibility to Ganciclovir induced cell death. Finally, a stereotactic biopsy of the left sided corpus callosum within a week of the [¹⁸F]FHBG PET scan confirmed the presence of malignant disease infiltrated with CD8+ T cells.

Discussion and Diagnosis

The patient was in his usual state of good health until he experienced his first grand mal seizure. The initial work-up following neurologic stabilization with Dilantin, failed to reveal a causative etiology. Brain imaging revealed a 2 cm enhancing right occipital mass consistent in extra-axial location and imaging attributes with a meningioma. The patient was diagnosed with an idiopathic seizure disorder; due to bone pain he was switched to Lamictal and had further seizure activity. Subsequently, the patient experienced new headaches with physical exertion and had an MRI for evaluation. The scan revealed a new small (currently available records do not document size) but enhancing mass in the right parietal occipital cortex that surgical exploration revealed was GBM, which was gross totally resected. The patient recovered without new neurological deficits and was started on radiation therapy, with an initial larger field receiving 50 Gray and a smaller coned down field in the involved region receiving 60 Gray of radiation exposure. The patient was administered 75 mg/m² daily doses of Temodar during radiation treatment followed by 6 months of adjuvant Temodar (200 mg/m²/day ×5 days every 4-weeks).

Treatment and Management

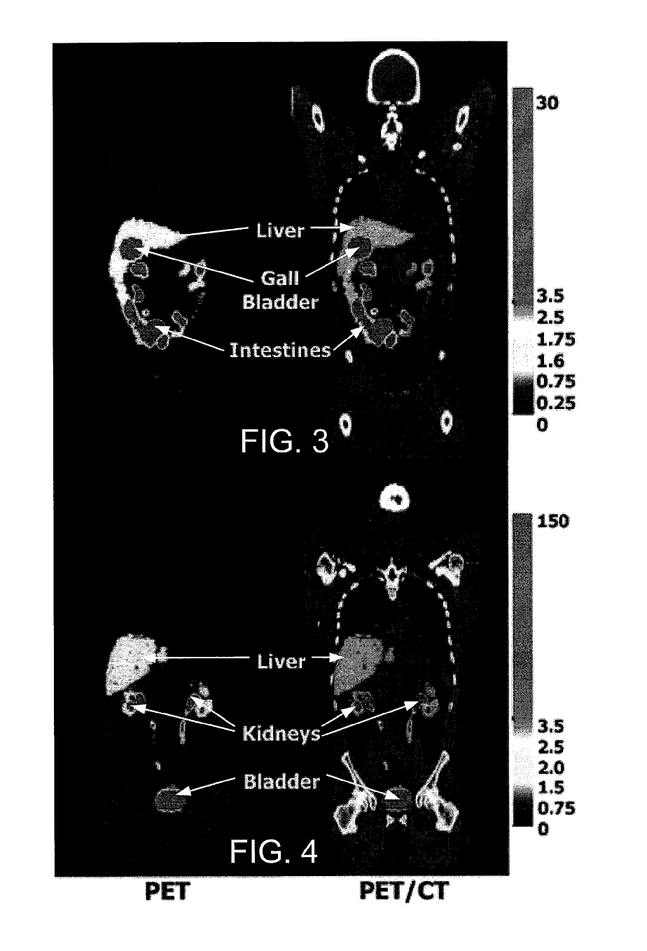

This example describes the only cancer patient, being treated with genetically modified cytolytic T cells that express HSV1-tk/Hygromycin (HyTK), who has to our knowledge ever been imaged with [¹⁸F]FHBG. In fact, this is believed to be the first report of imaging therapeutic cells in a human, using a reporter gene/probe technology. Previously, dendritic cells were imaged in human patients by directly labeling them ex vivo with superparamagnetic iron oxide particles or ¹¹¹In-Oxine ((2005) Nat. Biotechnol. 23, 1407-1413 and (1999) Cancer Res. 59, 56-58, both of which are incorporated herein by reference). The patient of the present example was enrolled in an ACGT clinical trial, receiving autologous CTLs expressing IL-13 zetakine and HSV1-tk, following surgical removal of his GBM tumor recurrence. IL-13 zetakine specifically targets CTLs to kill residual glioblastoma cells (((2004) Cancer Res. 64, 9160-9166, which is incorporated by reference)). HSV1-tk serves two purposes. As a safety gene, HSV1-tk expressing CTLs, exposed to the drug Ganciclovir, undergo programmed cell death. The HSV1-TK enzyme can also mono-phosphorylate [¹⁸F]FHBG, which can be used to image cells expressing the HSV1-tk PRG in living animals and humans using PET ((2007) Nat Protoc 1, 3069-3075, which is incorporated herein by reference). There are three currently conceived techniques for non-invasively imaging therapeutic cells in humans. The most conventional method is direct labeling of cells ex vivo with radionuclide or MRI probes, such as Indium-111 Oxine or Feridex (Berlex Laboratories, Wayne, N.J., USA). Easy implementation, reduced whole-body radiation exposure (radionuclide probes) and low signal to background ratio (if cells do not release the probe inside the patient) are these technique's advantages. Disadvantages include potential false positive images about cell location, lack of accurate information about cell survival, probe dilution after cell division and radionuclide probes' activity decay; hence this is primarily a short term monitoring technique. Another approach is detecting therapeutic cells with a very specific probe for a receptor found only on their surface. This is not a general method, requiring development of specific imaging probes for potentially every type of therapeutic cells. Even then, sensitivity may be low. The methods of the present example employ the reporter gene/probe based imaging technique. The techniques of embodiments of the present disclosure involve stable incorporation of a radionuclide based or MRI reporter gene, regulated by a strong constitutive promoter into therapeutic cells, prior to administration into patients. The reporter probe is then injected anytime thereafter to image therapeutic cell location(s) and survival, providing a general solution for long-term cell monitoring. Factors affecting the sensitivity of this technique may include the reporter probe's pharmacokinetics and the level of reporter gene expression per cell.

[¹⁸F]FHBG has been investigated in normal human volunteers ((2001) J Nucl Med 42, 1225-1234, which is incorporated herein by reference) and it is now being studied in glioma patients without ACGT. FHBG's safety in rats and rabbits has also been comprehensively assessed. FIGS. 3 and 4 illustrates two coronal slices of a control patient's whole-body [¹⁸F]FHBG biodistribution PET image. The highest [¹⁸F]FHBG activity can be observed in organs involved in its clearance (bladder, kidney, ureters, liver, gall bladder and intestines). [¹⁸F]FHBG clears very rapidly from all other tissues. One always observes a sharp exponential decline of [¹⁸F]FHBG activity in both patient's and normal human volunteer's blood through time. [¹⁸F]FHBG does not cross the blood brain barrier (BBB), however a slight increase in [¹⁸F]FHBG background accumulation is observed within intact glioma tumors or tumor resection sites of control patients (glioma patient's who were not administered CTLs), perhaps due to a compromised BBB. However, quantitative analysis shows greater than two times higher [¹⁸F]FHBG accumulations in the resection site and intact tumor of the CTL infused patient.

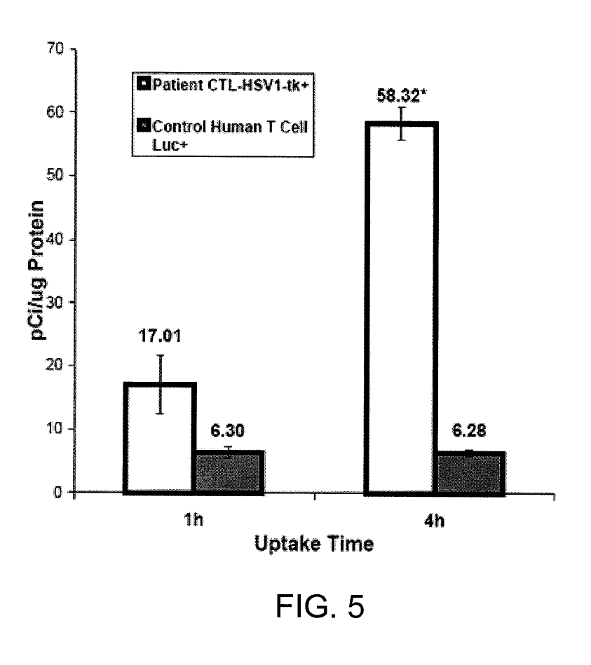

The patient of the present example received a total of approximately 1×10⁹ CTLs within a five-week period of direct infusions (one week break between weeks 2 and 4). It is both possible that these cells proliferate or some die once injected into the patient. The number of cells present within the tumor resection site and the intact tumor near the corpus callosum could not be specifically quantified. That would require a pharmacokinetic model ((2004) J Nucl Med 45, 1560-1570, which is incorporated herein by reference) for [¹⁸F]FHBG at brain tumor sites and knowing HSV1-tk expression levels per CTL at imaging time. The level of specific HSV1-TK activity in the cultured CTLs that were injected into the patient was analyzed by measuring the level of [³H]Penciclovir ([³H]PCV) uptake into the infused CTLs relative to control jurkat cells, which did not express HSV1-tk. FIG. 5 shows that at one and four hours, approximately 3× and 9×[³H]PCV, respectively, accumulated into the CTLs, relative to control cells. This low level of activity explains why [¹⁸F]FHBG accumulation in CTL infusion sites and intact tumor is only 2-3× higher than background. In fact during uptake assay cultured cells were exposed to a relatively constant concentration of about 1 μCi/ml; whereas assuming homogeneous distribution of [¹⁸F]FHBG in 6 liters of blood, the maximum concentration CTLs would have been exposed to within the first 1-2 minutes after injection would have been about 1 μCi/ml, but then there is a rapid decline in blood concentration of [¹⁸F]FHBG within the first 20-40 minutes following it's injection. Despite that, [¹⁸F]FHBG's ability to detect the CTLs may be due to the low background within the surrounding brain tissue and the presence of a large number of CTLs.

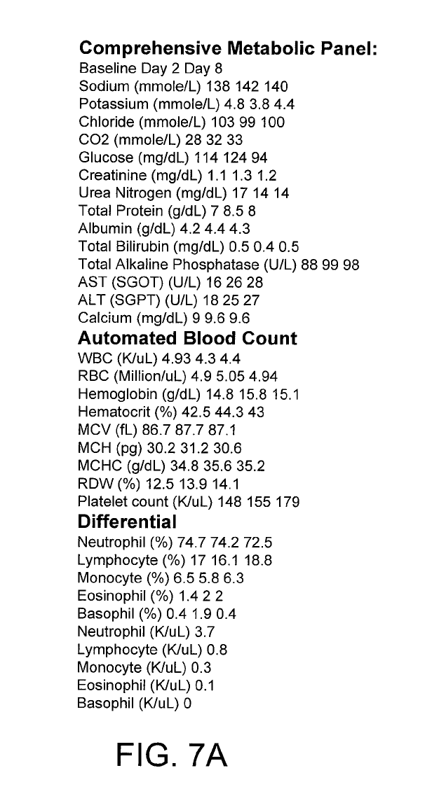

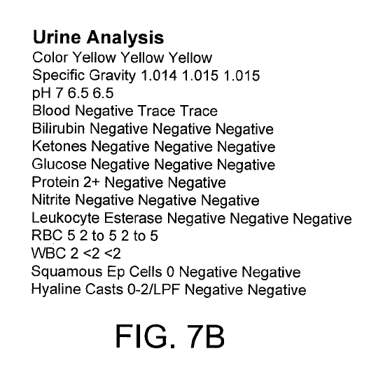

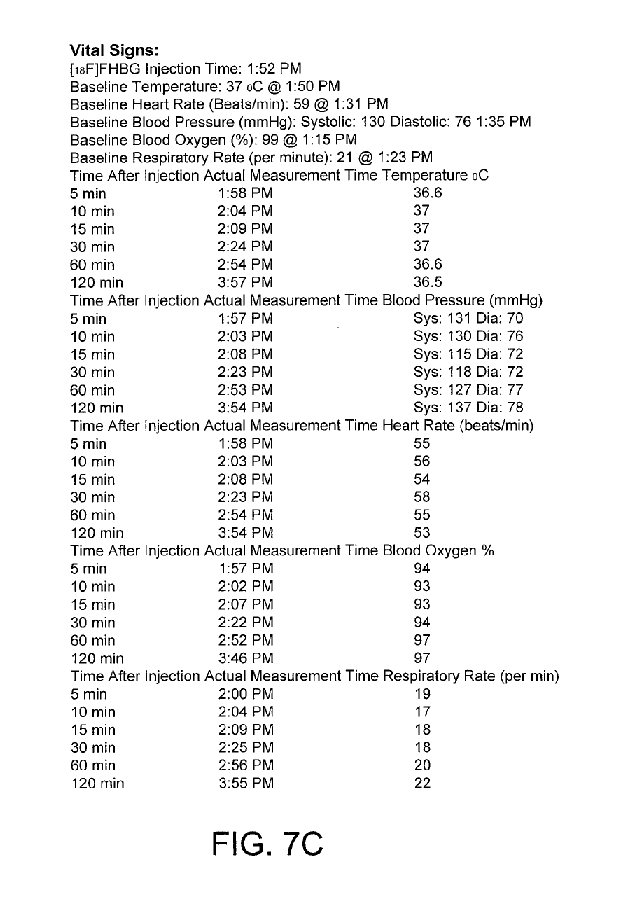

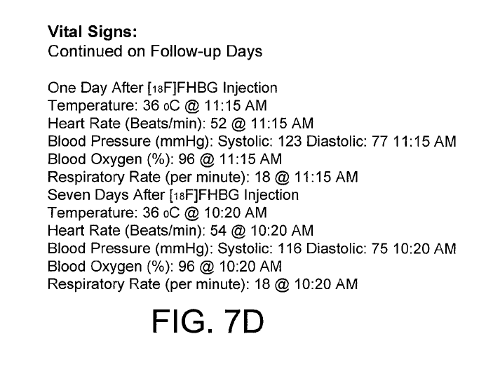

FIGS. 7A-7D illustrate tables including vital signs information about the patient.

FIG. 8 illustrates a table describing quality assurance criteria of infused CTLs.

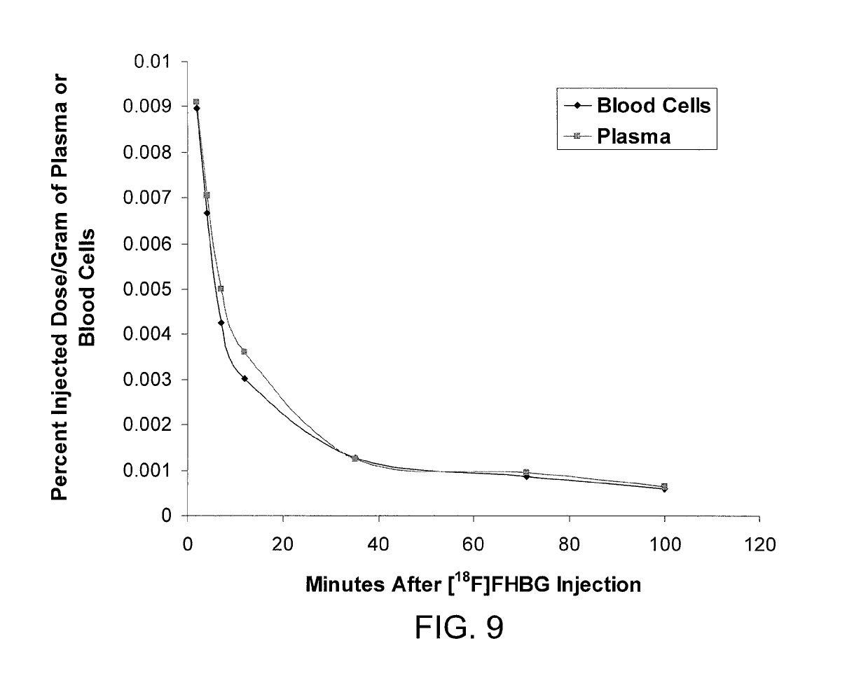

FIG. 9 illustrates a graph of the percent injected dose per gram of plasma or blood cells versus minutes after probe injection for blood cells and plasma.

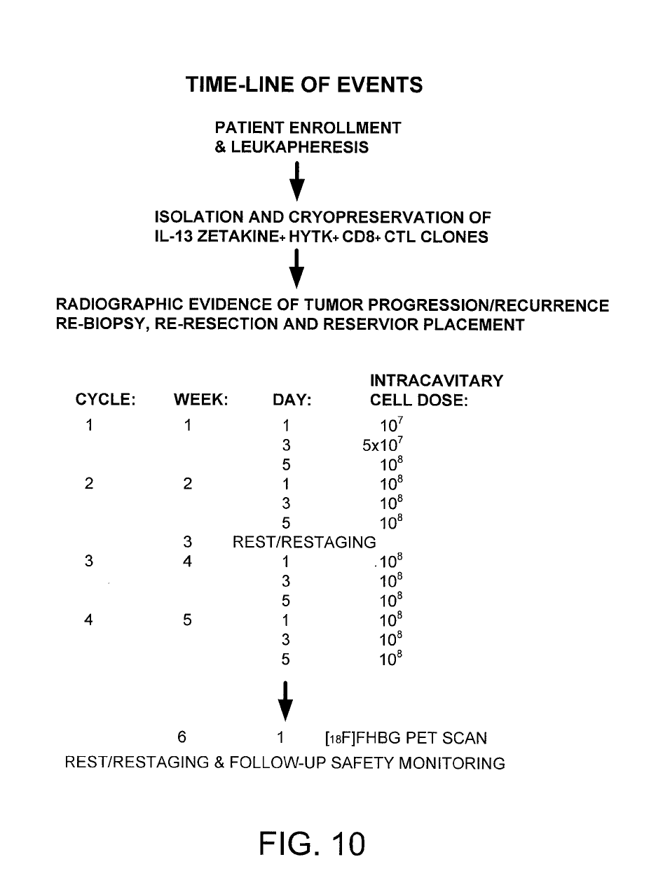

FIG. 10 illustrates a Time-Line of Events of the study.

### CONCLUSION

Glioblastoma multiforme (GM) is the most common and malignant primary brain tumor ((2007) Brain 130, 2596-2606, which is incorporated by reference). The median GM patient's survival is around 12 months and very few survive more than 3 years ((2007) Brain 130, 2596-2606, which is incorporated by reference). Therefore, ACGT is a much needed treatment that should be investigated for extending GM patient survival. Long term imaging of therapeutic cells will be important for predicting long-term efficacy of this type of treatment. This example is believed to be the first ever reported reporter gene based imaging of therapeutic cells in a human patient. This example also illustrates that [¹⁸F]FHBG, which normally cannot cross the blood brain barrier, can accumulate within glioma tumors and can detect HSV1-tk expressing therapeutic cells within these tumors.

Inclusion and Exclusion Criteria

Inclusion Criteria For Study Enrollment:
Histological verification of grade III or IV malignant glioma at original diagnosis.
Male or female subjects between 18-70 yrs, inclusive.
Unifocal site of original disease in the cerebral cortex.
Primary therapy completed and steroid independent no less than 4 weeks.
If patient is taking adjunct cytotoxic chemotherapy, patient must be at least two weeks from finishing most recent course and recovered from all acute side effects.
Adequate renal function as evidenced by creatinine <1.6.
Adequate bone marrow function as evidenced by WBC ≧2,000/d1 (or ANC>1,000) and platelets ≧100,000/dl unsupported by transfusion or growth factor
Normal liver function as evidenced by bilirubin <1.5, and SGOT and SGPT<2× upper limits of normal.
Female patients of childbearing potential must not be pregnant as evidenced by a serum β-HCG pregnancy test obtained within 7 days of enrollment.
Participants having reproductive potential must agree to use effective contraception during participation on this protocol

Exclusion Criteria For Study Enrollment:
Prior re-resection for recurrent/progressive disease.
Communication of resection cavity with ventricles/deep CSF pathways.
Survival expectation less than 3 months.
Karnofsky Performance Score (KPS)<70.
Organ Function: Pulmonary-Requirement for supplemental oxygen use that is not expected to resolve within 2 weeks, Cardiac-Uncontrolled cardiac arrhythmia, hypotension requiring pressor support, Renal-Dialysis dependent, Neurologic-refractory seizure disorder, clinically evident progressive encephalopathy.
Patients with any non-malignant intercurrent illness which is either poorly controlled with currently available treatment, or which is of such severity that the investigators deem it unwise to enter the patient on protocol shall be ineligible.
Patients being treated for severe infection or who are recovering from major surgery are ineligible until recovery is deemed complete by the investigator.
Failure to understand the basic elements of the protocol and/or the risks/benefits of participating in this pilot study.

History of Ganciclovir and/or Prohance contrast allergy or intolerance.

Positive serology for HIV based on testing performed within 3 months prior to protocol enrollment.

Nucleotide Sequence of HSV1-sr39tk:(SEQ ID NO: 3)

### Sr39TK (1128 bp)

ATG GCT TCG TAC CCC GGC CAT CAA CAC GCG TCT GCG TTC GAC CAG GCT GCG CGT TCT CGC GGC CAT AGC AAC CGA CGT ACG GCG TTG CGC CCT CGC CGG CAG CAA GAA GCC ACG GAA GTC CGC CCG GAG CAG AAA ATG CCC ACG CTA CTG CGG GTT TAT ATA GAC GGT CCC CAC GGG ATG GGG AAA ACC ACC ACC ACG CAA CTG

We claim the following:

1. A method of imaging the location and survival of an engineered cell in a human with an imaging reporter probe comprising:
delivering to the human subject an engineered cell, wherein the engineered cell includes an imaging reporter gene, and wherein the imaging reporter gene is expressed in the cell, thereby generating an imaging reporter gene product;
administering to the human subject an imaging reporter probe, wherein the imaging reporter probe freely enters and exits the cell, wherein the imaging reporter gene product interacts with the imaging reporter probe to form a modified imaging reporter probe, wherein the modified imaging reporter probe accumulates either within the engineered cell or on the surface of the engineered cell that expresses the imaging reporter gene, wherein the imaging reporter probe has a characteristic that it is capable of being imaged non-invasively using a nuclear imaging system, magnetic resonance imaging system, or an optical imaging system in the human; and
non-invasively imaging the human subject, wherein detecting the presence of the modified imaging reporter probe corresponds to the presence of the engineered cell.

2. The method of claim 1, wherein the imaging reporter probe is 9-[4-[¹⁸F]fluoro-3-(hydroxymethyl)butyl]guanine.

3. The method of claim 1, wherein the imaging reporter probe is a nuclear imaging probe that has the characteristic of being able to detect the expression of a nuclear imaging reporter gene.

4. The method of claim 1, wherein the imaging reporter probe is an MRI probe that has the characteristic of being able to detect the expression of a specific MRI based reporter gene.

5. The method of claim 1, wherein the reporter probe is an optical probe that has the characteristic of being able to detect the expression of an optical reporter gene.

6. The method of claim 1, wherein the engineered cell was genetically modified ex vivo prior to delivery to the human subject.

7. The method of claim 1, wherein the engineered cell has been genetically modified ex vivo to express an imaging reporter gene.

8. The method of claim 1, wherein the engineered cells is a cytolytic T cell.

9. The method of claim 8, wherein the cytolytic T cell is a cytolyltic CD8+ T cell.

10. The method of claim 1, wherein the imaging reporter gene is selected from a group consisting of: a HSV1-tk reporter gene and a HSV1-sr39tk PET reporter gene.

11. The method of claim 10, wherein the imaging reporter probe is a probe that has the characteristic of being able to detect the expression of a HSV1-tk or a HSV1-sr39tk PET reporter genes in cells within humans.

12. A method of imaging the location and survival of an engineered cell in a human comprising:
delivering to the human subject an engineered cell, wherein the engineered cell includes an imaging reporter gene, and wherein the imaging reporter gene is expressed in the engineered cell, thereby generating an imaging reporter gene product, wherein the imaging reporter gene product accumulates either within the engineered cell or on the surface of the engineered cell that expresses the imaging reporter gene, wherein the imaging reporter gene product has a characteristic that it is capable of being imaged non-invasively using a nuclear imaging system, magnetic resonance imaging system, or an optical imaging system in the human; and
non-invasively imaging the human subject, wherein detecting the presence of the imaging reporter gene product corresponds to the presence of the engineered cell.

13. The method of claim 12, wherein the reporter gene encodes a protein that is directly imageable.

14. The method of claim 13, wherein the protein is a bioluminescent protein.

15. A method of imaging the location and survival of an engineered cell in a human with an imaging reporter probe comprising:
delivering to the human subject an engineered cell, wherein the engineered cell includes an imaging reporter gene;
administering to the human subject an imaging reporter probe, wherein the imaging reporter probe freely enters and exits the cell, wherein the imaging reporter gene interacts with the imaging reporter probe so that the imaging reporter probe accumulates either within the engineered cell or on the surface of the engineered cell, wherein the imaging reporter probe has a characteristic that it is capable of being imaged non-invasively using a nuclear imaging system, magnetic resonance imaging system, or an optical imaging system in the human; and
non-invasively imaging the human subject, wherein detecting the presence of the imaging reporter probe corresponds to the presence of engineered cell.

16. A kit comprising:
an engineered cell, wherein the engineered cell includes an imaging reporter gene, and wherein the imaging reporter gene is expressed in the cells, thereby generating an imaging reporter gene product;
an imaging reporter probe; and
directions for use.

17. The kit of claim 16, wherein the imaging reporter gene is selected from a group consisting of a HSV1-tk reporter gene and a HSV1-sr39tk PET reporter gene, wherein the imaging reporter probe is 9-[4-[¹⁸F]fluoro-3-(hydroxymethyl)butyl]guanine, and wherein the engineered cells are cytolyltic CD8+ T cells.

18. A kit comprising:
an engineered cell, wherein the engineered cell includes an imaging reporter gene, and wherein the imaging reporter gene is expressed in the cells, thereby generating an imaging reporter gene product, wherein the imaging reporter gene product accumulates either within the cells or on the surface of the cells that express the imaging reporter gene, wherein the imaging reporter gene product has a characteristic that it is capable of being imaged non-invasively using a nuclear imaging system, magnetic resonance imaging system, or an optical imaging system in the human; and directions for use.

19. An engineered cell, comprising:
an imaging reporter gene, wherein the imaging reporter gene is expressed in the cells, thereby generating an imaging reporter gene product.

20. The engineered cell of claim 19, wherein the imaging reporter gene is selected from a group consisting of: a HSV1-tk reporter gene and a HSV1-sr39tk PET reporter gene, wherein the imaging reporter gene produces an imaging reporter gene product that interacts with an imaging reporter probe, and wherein the imaging reporter probe is 9-[4-[¹⁸F]fluoro-3-(hydroxymethyl)butyl]guanine, and wherein the engineered cells are cytolyltic CD8+ T cells.

21. An engineered cell, comprising:
an imaging reporter gene, and wherein the imaging reporter gene is expressed in the cells, thereby generating an imaging reporter gene product, wherein the imaging reporter gene product accumulates either within the cells or on the surface of the cells that express the imaging reporter gene, wherein the imaging reporter gene product has a characteristic that it is capable of being imaged non-invasively using a nuclear imaging system, magnetic resonance imaging system, or an optical imaging system in the human.

22. A method of imaging the location and survival of an engineered cell in a host with an imaging reporter probe comprising:
delivering to the host an engineered cell, wherein the engineered cell includes an imaging reporter gene, and wherein the imaging reporter gene is expressed in the cell, thereby generating an imaging reporter gene product;
administering to the host an imaging reporter probe, wherein the imaging reporter probe freely enters and exits the cell, wherein the imaging reporter gene product interacts with the imaging reporter probe to form a modified imaging reporter probe, wherein the modified imaging reporter probe accumulates either within the engineered cell or on the surface of the engineered cell that expresses the imaging reporter gene, wherein the imaging reporter probe has a characteristic that it is capable of being imaged non-invasively using a nuclear imaging system, magnetic resonance imaging system, or an optical imaging system in the host; and
non-invasively imaging the host, wherein detecting the presence of the modified imaging reporter probe corresponds to the presence of the engineered cell.

23. A method of imaging the location and survival of an engineered cell in a host comprising:
delivering to the host an engineered cell, wherein the engineered cell includes an imaging reporter gene, and wherein the imaging reporter gene is expressed in the engineered cell, thereby generating an imaging reporter gene product, wherein the imaging reporter gene product accumulates either within the engineered cell or on the surface of the engineered cell that expresses the imaging reporter gene, wherein the imaging reporter gene product has a characteristic that it is capable of being imaged non-invasively using a nuclear imaging system, magnetic resonance imaging system, or an optical imaging system in the host; and
non-invasively imaging the host, wherein detecting the presence of the imaging reporter gene product corresponds to the presence of the engineered cell.

24. A method of imaging the location and survival of an engineered cell in a host with an imaging reporter probe comprising:
delivering to the host an engineered cell, wherein the engineered cell includes an imaging reporter gene;
administering to the host an imaging reporter probe, wherein the imaging reporter probe freely enters and exits the cell, wherein the imaging reporter gene interacts with the imaging reporter probe so that the imaging reporter probe accumulates either within the engineered cell or on the surface of the engineered cell, wherein the imaging reporter probe has a characteristic that it is capable of being imaged non-invasively using a nuclear imaging system, magnetic resonance imaging system, or an optical imaging system in the host; and
non-invasively imaging the host, wherein detecting the presence of the imaging reporter probe corresponds to the presence of engineered cell.
